(19) United States
(12) Patent Application Publication
(10) Pub. No.: US 2025/0064635 A1
(43) Pub. Date: Feb. 27, 2025

(54) DEVICES AND METHODS FOR POSTERIOR EYE SEGMENT ACCESS WITH ACCURATE LOCALIZATION AND NEEDLE PENETRATION DEPTH

(71) Applicants: Precision Retina Inc., Toronto (CA); Unity Health Toronto, Toronto (CA)

(72) Inventors: Rajeev Muni, Toronto (CA); Aaron Phillip Weinroth, Thornhill (CA)

(21) Appl. No.: 18/770,797

(22) Filed: Jul. 12, 2024

Related U.S. Application Data
(63) Continuation of application No. PCT/CA2024/050924, filed on Jul. 11, 2024.
(60) Provisional application No. 63/578,330, filed on Aug. 23, 2023.
(60) Provisional application No. 63/624,372, filed on Jan. 24, 2024.

(51) Int. Cl. A61F 9/00; A61F 9/007; A61M 5/32

(52) U.S. Cl. CPC A61F 9/0017; A61F 9/00736; A61M 5/3134; A61M 5/3287; A61M 5/46; A61M 5/16804; A61M 5/31533; A61M 2205/581; A61M 2205/587; A61M 2210/0612

(57) ABSTRACT

Methods and devices are described herein for a) injecting/delivering fluid to and/or draining/removing fluid from the eye. One of the devices may comprise a probe including a main body having a distal end portion; a needle that extends and retracts from an exit location on a side surface of the distal end portion, the needle having a needle conduit; and one or more probe conduits for moving the fluid through the probe, the one or more probe conduits being fluidically coupled to the needle conduit. During use, a portion of the side surface having the exit location is placed adjacent to a surface of the eye and the needle is extended to penetrate into the eye, and the fluid is injected or drained through the needle conduit.

### CROSS-REFERENCE TO RELATED APPLICATIONS

This application is a continuation of International Application No. PCT/CA2024/050924 filed Jul. 11, 2024, which claims priority from U.S. Provisional Patent Application No. 63/578,330 filed Aug. 23, 2023, and from U.S. Provisional Patent Application No. 63/624,372 filed Jan. 24, 2024; the entire contents of each of which are hereby incorporated herein by reference in their entirety.

### FIELD

The various embodiments described herein generally relate to a device and method for delivery of agents (including drugs) to the posterior segment of the eye (suprachoroidal space or subretinal space) via direct external scleral penetration with accurate localization and precise needle depth penetration such as for minimally invasive suprachoroidal delivery of a viscoelastic agent for repair of a retinal tear or rhegmatogenous retinal detachment.

### BACKGROUND

Rhegmatogenous retinal detachment (RRD) repair has evolved tremendously during the past century. Although scleral buckle (SB) was the dominant technique for many decades¹, since the early 21^{st} century, pars plana vitrectomy (PPV) has been the treatment of choice for most surgeons². However, the functional outcomes after PPV have been reported to be inferior to SB³ and pneumatic retinopexy⁴. Advances in multimodal imaging have demonstrated a high risk of unwanted structural abnormalities after PPV⁵⁻⁸. Recent evidence suggests that additional procedures, such as sub-retinal fluid drainage⁹, use of heavy liquids¹⁰, and large gas tamponades¹¹, may be harmful in some cases. This knowledge has led surgeons to modify techniques to not only achieve single-operation reattachment but also maximize the integrity of reattachment.

Some conventional techniques of suprachoroidal delivery of a viscoelastic agent for repair of a retinal tear or retinal detachment have involved a scleral cutdown (e.g., incision into the sclera) with or without tissue dissection (manual separation of choroid from sclera) and direct injection of a viscoelastic agent, or a cutdown followed by the passage of a probe into and through the suprachoroidal space and injection of a viscoelastic agent once the probe is in the region of the retinal tear. However, these are relatively invasive procedures (with potentially greater risk of hemorrhage and other complications) that must be performed in an operating room, which increases cost and delay for when an operating room is available for use. Accordingly, there is a need for a minimally invasive technique which does not necessarily have to be performed in the operating room and if performed in an operating room can be performed without a scleral incision.

Similarly, techniques for delivery of therapeutic agents such as drugs to the retina and other structures toward the back of the eye, including in the subretinal space, are either invasive (such as with ocular incisions and/or subretinal injections) or untargeted (such as with intravitreal injections) which results in risks of complications such as damage to the eye, compromised vision, and/or dilution of the therapeutic effect. Accordingly, there is also a need for a minimally invasive technique for targeted delivery of therapeutic agents toward the back of the eye via the suprachoroidal and/or subretinal spaces.

### SUMMARY OF VARIOUS EMBODIMENTS

In one aspect, in accordance with the teachings herein, there is provided at least one embodiment of a device for injecting or draining a fluid into or from an eye, wherein the device comprises: a probe comprising: a main body having a distal end portion; a needle that extends and retracts from an exit location on a side surface of the distal end portion, the needle having a needle conduit; and one or more probe conduits for moving the fluid through the probe, the one or more probe conduits being fluidically coupled to the needle conduit; wherein during use, a portion of the side surface having the exit location is placed adjacent to a surface of the eye and the needle is extended to penetrate into the eye, and the fluid is injected or drained through the needle conduit.

In at least one embodiment, the needle is configured to exit the probe substantially perpendicular to a tangent of the side surface at the exit location.

In at least one embodiment, the side surface is concave with a radius of curvature that approximately matches a radius of curvature of the sclera.

In at least one embodiment, the longitudinal axis of the distal end portion is at an angle to a longitudinal axis of the main body.

In at least one embodiment, the device comprises a needle actuator that is coupled to the needle and controllable for causing the needle to extend and retract.

In at least one embodiment, the device comprises a fluid actuator that is coupled to the needle and controllable for causing the fluid to move between the one or more probe conduits and the eye through the needle conduit.

In at least one embodiment, the side surface has a boss at the exit location and the needle is configured to extend and retract through the boss, or the side surface has a boss adjacent the exit location and the needle is configured to extend and retract adjacent to the boss.

In at least one embodiment, the one or more probe conduits comprise an injection conduit and a drainage conduit, and the probe has a coupling that is adjustable between fluidically coupling the drainage conduit to the needle conduit and fluidically coupling the injection conduit to the needle conduit.

In at least one embodiment, the device further comprises a guidance light source that is adapted to generate a guidance light beam for illumination or to indicate when a tip of the needle penetrates into different layers of the eye by changes in transmitted or reflected light.

In at least one embodiment, the device further comprises at least one guidance tool that is adapted to perform a measurement to determine a position of a tip of the needle and/or a target injection or drainage site in the eye.

In at least one embodiment, the device further comprises a control unit that is contained in the probe or remote from the probe, the control unity comprising: a display that is optional; a memory unit for storing software instructions for performing one or more functions; a device interface for receiving measurement data and transmitting control signals for operation of the device; a speaker or vibrator to generate audio signals or vibrations corresponding to device operating parameters and/or the measurement data, where the speaker or vibrator are optional; a processor that is communicatively coupled to any of the memory unit, the interface, the speaker or vibrator, and the display, the processor being configured to perform the one or more functions when executing the software instructions, the one or more functions including: receiving the measurement data; transmitting the control signals; generating the audio signals or vibrations; and displaying at least a portion of the measurement data on the display; and a power source for providing power to components of the device.

In at least one embodiment, the device includes a pump fluidically coupled to the one or more probe conduits that is controllable to create an injection pressure when the fluid is injected into the eye or a drainage pressure when the fluid is drained from the eye.

In at least one embodiment, the probe further comprises a flange and/or a variable coupler at the exit location to maintain a position or a pressure between the side surface and the surface of the eye.

In at least one embodiment, the flange and/or the variable coupler further comprises one or more sensors to measure a position and/or a pressure at one or more points between the side surface of the distal end portion of the probe and the surface of the eye.

In at least one embodiment, the device further comprises an injection fluid container and/or a drainage fluid container coupled to the one or more probe conduits.

In at least one embodiment, the needle is adapted to extend to a depth within a suprachoroidal, subretinal, or intravitreal space of the eye.

In at least one embodiment, when the eye has a rhegmatogenous retinal detachment (RRD) or a retinal tear and the device is adapted for injecting the fluid into a suprachoroidal space of the eye to create a choroidal buckle for treating the RRD or the retinal tear.

In at least one embodiment, the fluid comprises a treatment fluid including any combination of a drug, a gene therapy, an extended release implant, a viscoelastic, a hydrogel, and a gas.

In another aspect, in accordance with the teachings herein, there is provided a method for injecting or draining a fluid into or from an eye, wherein the method comprises: placing a side surface of a distal end portion of a probe adjacent to a surface of the eye, the probe having a needle with a needle conduit and the needle is retracted; extending the needle from an exit location on the side surface of the distal end portion of the probe to penetrate the eye; and injecting or draining fluid between the probe and the eye through the needle conduit.

In at least one embodiment, the method comprises extending the needle substantially perpendicular to a tangent of the side surface of the probe at the exit location.

In at least one embodiment, the side surface of the distal end portion of the probe is concave with a radius of curvature that approximately matches a radius of curvature of the sclera.

In at least one embodiment, a longitudinal axis of the distal end portion is at an angle to a longitudinal axis of the main body.

In at least one embodiment, the method comprises using a needle actuator for controlling extension and retraction of the needle.

In at least one embodiment, the method comprises using a fluid actuator for controlling injection and drainage of the fluid.

In at least one embodiment, the method comprises using a guidance light beam and/or a measurement made by a guidance tool to determine a position of a tip of the needle and/or a target injection or drainage site in the eye.

In at least one embodiment, a control unit that is integral with or separate from the probe is used to display measurement data from the probe, transmit control signals to the probe, and/or generate audio signals or vibrations corresponding to device operating parameters and/or the measurement data.

In at least one embodiment, the method comprises extending the needle into a suprachoroidal, a subretinal, or an intravitreal space of the eye.

In at least one embodiment, the method comprises using a fluid that comprises a treatment fluid including any combination of a drug, a gene therapy, an extended release implant, a viscoelastic, a hydrogel, and a gas.

In at least one embodiment, the eye has a rhegmatogenous retinal detachment (RRD) or a retinal tear and the method comprises injecting the fluid is into a suprachoroidal space (SCS) of the eye to create a choroidal buckle for treating the RRD or the retinal tear.

It will be appreciated that the foregoing summary sets out representative aspects of embodiments to assist skilled readers in understanding the following detailed description. Other features and advantages of the present application will become apparent from the following detailed description taken together with the accompanying drawings. It should be understood, however, that the detailed description and the specific examples, while indicating preferred embodiments of the application, are given by way of illustration only, since various changes and modifications within the spirit and scope of the application will become apparent to those skilled in the art from this detailed description.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1 is a diagram of an example embodiment of an ocular treatment device for accurate localization and precise needle depth penetration for ocular treatments such as treating retinal tear or rhegmatogenous retinal detachment (RRD), or for delivering a drug, other therapeutic agent or other treatment fluid, for example, in accordance with the teachings herein.

FIG. 2A is a diagram of an example embodiment of an alternative probe that can be used with an ocular treatment device for accurate localization and precise needle depth penetration for ocular treatments such as for treating retinal tear or RRD, or for delivering a drug, other therapeutic agent or other treatment fluid, for example, in accordance with the teachings herein.

FIG. 2B is a diagram of an example embodiment of a control unit to be used with the ocular treatment device in accordance with the teachings herein.

FIG. 2C is a block diagram of an example embodiment of various components of the control unit of FIG. 2B.

FIG. 3A shows several embodiments of a distal end of probes with varying curvatures and length for localization at different locations on the eye.

FIG. 3B shows a magnified view of a distal end of one of the probes of FIG. 3A.

FIGS. 3C-3D show examples of placement of the distal end of the probe at different locations on the eye, with needle penetration and fluid injection at different depths.

FIG. 3E shows an example embodiment of a portion of an ocular treatment device with a pressure distribution flange.

FIG. 3F shows an example embodiment of a portion of an ocular treatment device a pressure distribution flange and a variable coupler.

FIG. 3G shows a front view of an example embodiment of the pressure distribution flange with one or more sensors.

FIG. 3H shows an example embodiment of an alternative probe that can be used with an ocular treatment device for accurate localization and precise needle depth penetration for ocular treatments where the probe is a standalone device.

FIG. 4A is a flowchart of an example embodiment of a method for treating retinal tear or RRD in accordance with the teachings herein.

FIG. 4B is a flowchart of another example embodiment of a method for treating retinal tear or RRD in accordance with the teachings herein.

FIGS. 4C-4N show images at different stages of a method for repairing a retinal tear or RRD.

FIG. 4O shows a flowchart of an example embodiment of a method for precise localization and depth penetration on a surface of the eye for an ocular procedure.

FIGS. 5A-5C show longitudinal ultrawide-field photographs of a patient with pseudophakia presenting with a RRD in the right eye.

FIG. 6 shows a final appearance of the choroidal convexity formed after a Suprachoroidal Treatment (ST) procedure in accordance with the teachings herein.

FIGS. 7A-7B are longitudinal vertical swept-source optical coherence tomography (SS-OCT) scans at the ST injection site taken at postoperative day 1 and postoperative day 5, respectively, after the ST procedure.

FIGS. 8A-8D show a baseline longitudinal SS-OCT scan, a postoperative day 1 SS-OCT scan, a postoperative day 2 SS-OCT scan and a postoperative day 3 SS-OCT scan, respectively, after the ST procedure.

FIG. 9 shows a fundus autofluorescence image on postoperative day 5 after the ST procedure.

FIGS. 10A-10C show longitudinal SS-OCT scans at the temporal macula and temporal mid-periphery demonstrating the location where the ST procedure was performed (at the left side of the image) on postoperative day 1, postoperative day 3 and postoperative day 5, respectively, after the ST procedure.

FIGS. 11A-11B are OCT scan images showing a hyporeflective space between the choroid and sclera (arrowheads) and a mild residual inferior subretinal fluid with no outer retinal folds in the extreme inferior periphery (star), respectively, after the ST procedure.

Further aspects and features of the example embodiments described herein will appear from the following description taken together with the accompanying drawings.

### DETAILED DESCRIPTION OF THE EMBODIMENTS

Various embodiments in accordance with the teachings herein will be described below to provide examples of at least one embodiment of the claimed subject matter. No embodiment described herein limits any claimed subject matter. The claimed subject matter is not limited to devices, systems or methods having all of the features of any one of the devices, systems or methods described below or to features common to multiple or all of the devices, systems or methods described herein. It is possible that there may be a device, system or method described herein that is not an embodiment of any claimed subject matter. Any subject matter that is described herein that is not claimed in this document may be the subject matter of another protective instrument, for example, a continuing patent application, and the applicants, inventors or owners do not intend to abandon, disclaim or dedicate to the public any such subject matter by its disclosure in this document.

Furthermore, it will be appreciated that for simplicity and clarity of illustration, where considered appropriate, reference numerals may be repeated among the figures to indicate corresponding or analogous elements or steps. In addition, numerous specific details are set forth in order to provide a thorough understanding of the embodiments described herein. However, it will be understood by those of ordinary skill in the art that the embodiments described herein may be practiced without these specific details. In other instances, well-known methods, procedures and components have not been described in detail so as not to obscure the embodiments described herein. Also, the description is not to be considered as limiting the scope of the embodiments described herein.

It should also be noted that the terms “coupled” or “coupling” as used herein can have several different meanings depending on the context in which these terms are used. For example, the terms coupled or coupling can have a mechanical, electrical or communicative connotation. For example, as used herein, the terms coupled or coupling can indicate that two elements or devices can be directly connected to one another or connected to one another through one or more intermediate elements or devices via an electrical element, an electrical signal, a light signal or a mechanical element depending on the particular context.

Similarly, throughout this specification and the appended claims the term “communicative” as in “communicative pathway”, “communicative coupling”, and in variants such as “communicatively coupled” is generally used to refer to any engineered arrangement for transferring and/or exchanging information. Examples of communicative pathways include, but are not limited to, electrically conductive pathways (e.g., electrically conductive wires, physiological signal conduction), electromagnetically radiative pathways (e.g., radio waves, optical signals, etc.), or any combination thereof. Examples of communicative couplings include, but are not limited to, electrical couplings, magnetic couplings, radio couplings, optical couplings or any combination thereof.

Unless the context requires otherwise, throughout the specification and claims which follow, the word “comprise” and variations thereof, such as, “comprises” and “comprising” are to be construed in an open, inclusive sense, that is, as “including, but not limited to”.

It should also be noted that, as used herein, the wording “and/or” is intended to represent an inclusive-or. That is, “X and/or Y” is intended to mean X or Y or both X and Y, for example. As a further example, the phrases “X, Y, and/or Z”, “any operable combination of X, Y and Z” or “X, Y, Z or any combination thereof” is intended to mean X, Y, Z, X and Y, X and Z, Y and Z, or X, Y and Z.

It should be noted that terms of degree such as “substantially”, “about” and “approximately” as used herein mean a reasonable amount of deviation of the modified term such that the end result is not significantly changed. These terms of degree may also be construed as including a deviation of the modified term, such as by 1%, 2%, 5%, 10% or 15%, for example, if this deviation does not negate the meaning of the term it modifies.

Furthermore, the recitation of numerical ranges by endpoints herein includes all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed, such as 1%, 2%, 5%, 10% or 15%, for example.

A portion of the example embodiments of the systems, devices, or methods described in accordance with the teachings herein may be implemented as a combination of hardware and/or software. For example, a portion of the embodiments described herein may be implemented, at least in part, by using one or more computer programs, executing on one or more programmable devices comprising at least one processing element, and at least one data storage element (including volatile and/or non-volatile memory). These devices may also have at least one input device (e.g., a keyboard, a mouse, a touchscreen, button, switches, dials, sliders and the like) and at least one output device (e.g., a display screen, a printer, a wireless radio, a speaker, a vibrator and the like) depending on the nature of the device.

It should also be noted that there may be some elements that are used to implement at least part of the embodiments described herein that may be implemented via software that is written in a high-level procedural language such as object-oriented programming. The program code may be written in C, C++ or any other suitable programming language and may comprise modules or classes, as is known to those skilled in object-oriented programming. Alternatively, or in addition thereto, some of these elements implemented via software may be written in assembly language, machine language, or firmware as needed.

At least some of the software programs used to implement at least one of the embodiments described herein may be stored on a storage media or a device that is readable by a general or special purpose programmable device. The software program code, when read by the programmable device, configures the programmable device to operate in a new, specific and predefined manner in order to perform at least one of the methods described herein.

Furthermore, at least some of the programs associated with the systems and methods of the embodiments described herein may be capable of being distributed in a computer program product comprising a computer readable medium that bears computer usable instructions, such as program code, for one or more processors. The program code may be preinstalled and embedded during manufacture and/or may be later installed as an update for an already deployed computing system. The medium may be provided in various forms, including non-transitory forms such as, but not limited to, one or more diskettes, compact disks, tapes, chips, and magnetic and electronic storage. In alternative embodiments, the medium may be transitory in nature such as, but not limited to, wire-line transmissions, satellite transmissions, internet transmissions (e.g., downloads), media, digital and analog signals, and the like. The computer useable instructions may also be in various formats, including compiled and non-compiled code.

Any module, unit, component, server, computer, terminal or device described herein that executes software instructions in accordance with the teachings herein may include or otherwise have access to computer readable media such as storage media, computer storage media, or data storage devices (removable and/or non-removable) such as, for example, magnetic disks, optical disks, or tape. Computer storage media may include volatile and non-volatile, removable and non-removable media implemented in any method or technology for storage of information, such as computer readable instructions, data structures, program modules, or other data. Examples of computer storage media include RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information, and which can be accessed by an application, module, or both. Any such computer storage media may be part of the device or accessible or connectable thereto.

Described herein are various example embodiments of methods and ocular treatment devices that may be used for accessing portions of the eye, such as the suprachoroidal and subretinal spaces, for example, from the external sclera with accurate localization and precise needle depth penetration for a variety of ocular treatments such as, but not limited, to injecting or draining fluidic substances including minimally invasive treatment of a retinal tear or RRD by delivery of a treatment agent, such as a viscoelastic substance, with the goal of creating a temporary choroidal buckle, for example. For example, a technique herein referred to as the ST procedure may be performed which includes the delivery of a treatment fluid, such as a viscoelastic agent, for example, into the suprachoroidal space for RRD repair. Various devices are described herein for performing the ST technique and providing the delivery of treatment fluid in a more predictable fashion. In another embodiment, an ocular treatment including the ST procedure may additionally include the aspiration of fluid from a patient's eye, such as subretinal fluid/hemorrhage or suprachoroidal fluid/hemorrhage, for example. In another embodiment, an ocular treatment performed independent of the ST procedure may include the aspiration of fluid from a patient's eye.

The various embodiments described herein are created to be minimally invasive so that the ocular procedures/treatments can be performed in a minimal medical setting such as a doctor's office but also in the operating room depending on surgeon comfort and depending on target location of the injection (for example very posterior locations such as in the macula may require a conjunctival cutdown and may preferably be performed in the operating room with wide-field viewing). However, these procedures/treatments may also be performed in other medical settings such as an ambulatory clinic. This is because the various embodiments described herein that provide access and precise localization advantageously allow for using a needle for removing fluid from the eye and/or injecting fluid, such as a viscoelastic agent or a drug, in the SCS or other region of the eye without the more invasive cutdown/catheter approach. Advantageously, since the devices and methods described herein provide accurate ocular localization on the external sclera and precise needle depth penetration this simplifies performing various ocular treatments which may reduce the risk of performing these ocular treatments and may improve procedure success and patient outcomes. Furthermore, since the accurate localization and needle depth precision techniques described herein allow for needle insertions, which is relatively non-invasive compared to surgery, this may allow patients to have a faster recovery with little to no downtime with restrictions on activity. This is less likely with conventional operating room techniques that are more invasive.

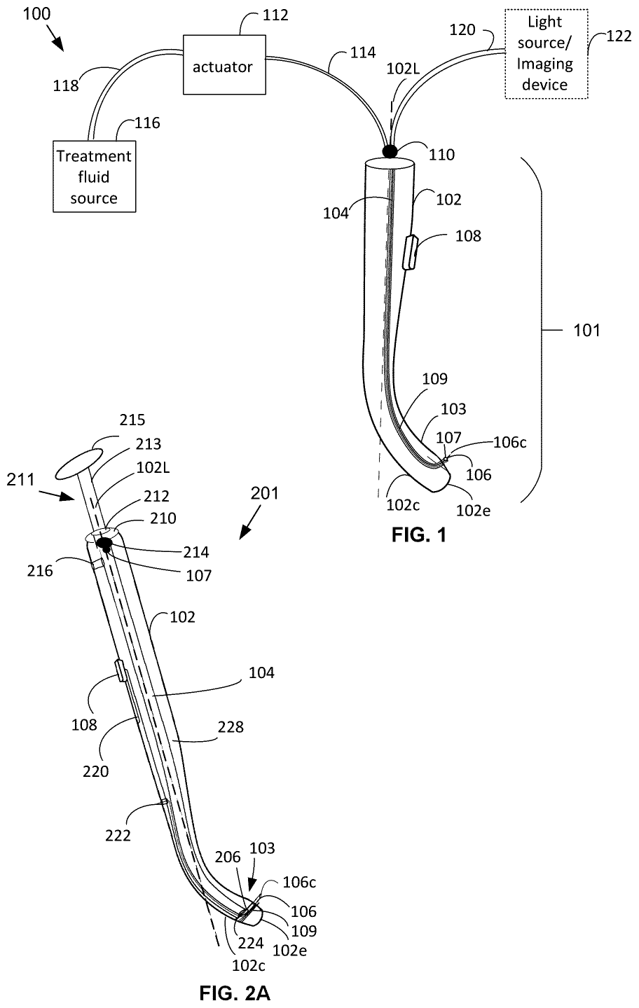

Referring now to FIG. 1, shown therein is an example embodiment of an ocular treatment device 100 for performing various ocular procedures including treating retinal tear or RRD, in accordance with the teachings herein. The ocular treatment device 100 includes a probe 101 and a fluid actuator 112. The probe 101 includes a main body 102, a conduit 104 which may be referred to as an injection conduit, and a needle 106. The main body has a longitudinal axis 102L and a distal end portion 103 that includes a curved portion 102c with respect to the longitudinal axis 102L. The longitudinal axis of the distal end portion 103 may be at an angle with respect to the longitudinal axis 102L of the probe 101, or it may be straight (i.e. parallel with the longitudinal axis of the probe). Also, in at least one embodiment, the distal end portion 103 may be curved according to a radius of curvature, only slightly curved or straight, (or at only a very shallow angle) such as when performing localization on more anterior scleral locations. The injection conduit 104 is adapted to receive a fluid, such as a treatment fluid, for injection into a region of the eye, such as the suprachoroidal space (SCS) of the eye of a patient for treating a retinal detachment or a retinal tear of the patient, or for injecting a therapeutic agent into a region of the eye such as the SCS or the subretinal space for treatment of a retinal or other ocular condition. The needle 106 is disposed at the distal end of the probe 101 and has a needle conduit 106c that is fluidically coupled to the injection conduit 104 for injecting the treatment fluid into the SCS of the eye or depending on the ocular treatment in other locations of the eye such as the subretinal space, the choroid or intravitreal. The proximal end of the needle conduit 106c is fluidically coupled with the injection conduit 104.

During use, the tip of the needle 106 and correspondingly the distal end of the needle conduit 106c is placed at the injection site, such as within the SCS of the patient's eye. Accordingly, the needle 106 is preferably disposed along the distal end 102c of the probe 101, preferably on a side surface of the probe and not at the end tip (i.e. not at the most distal end surface of the distal end of the probe), so that the needle 106 is approximately perpendicular/substantially perpendicular to a tangent of the surface of the sclera (and also approximately perpendicular to the longitudinal axis of the distal end portion 103) prior to being inserted into the sclera, which means that the needle 106 will be inserted along a direction generally pointing towards the centre of the globe (e.g., center of the patient's eye). Accordingly, the needle 106 is preferably retracted so that it does not extend past the surface of the distal end portion of the probe that is placed adjacent the eye. One the needle 106 is at the proper location, it can be then extended such that it extends from an exit location on the surface of the distal end portion of the probe into the eye. There may be a small aperture at the exit location that is located to surround the needle 106 when the needle 106 is extended so that the needle is substantially perpendicular to a tangent of a side surface of the distal end portion of the probe at the exit location. Furthermore, in at least one embodiment, guidance mechanisms and/or motorized/manual graduated advancement of the needle may be used for proper insertion of the needle 106 into the sclera and advancement to a desired depth, such as the SCS, for example. This allows the depth of the insertion of the needle tip to be more precisely known since the needle 106 is penetrating the sclera at the same depth as the needle length as well as for other reasons described below. The needle 106 can be selected from any Gauge needle but preferably from 21-31 Gauge needles such as a 30 Gauge needle or a 27 Gauge needle, for example.

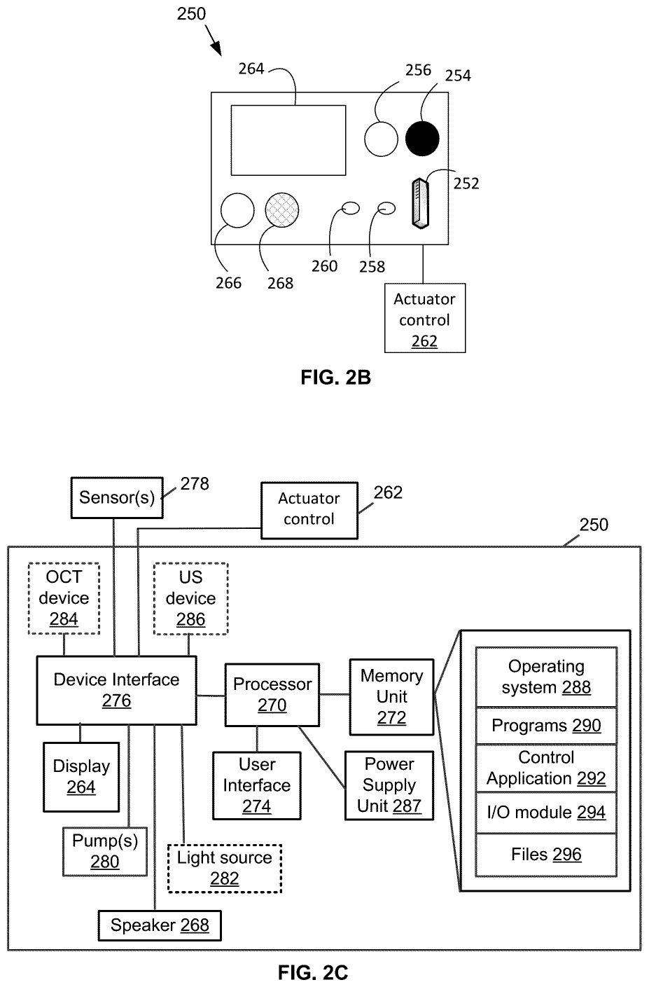

The probe 101 has a form factor that allows a proximal portion (e.g., upper portion when the probe is vertical as shown in FIGS. 1 and 2A) of it to be handheld. The distal portion 102c of the probe has a similar shape of the “business end” of a scleral depressor or a larger bulbous tip and a longitudinal axis of the distal portion 102c may be angled relative to the longitudinal axis 102L. The bulbous tip may have a thickness of about 3 mm to about 30 mm for example. For example, the width of the distal end of the probe that is placed adjacent to the patient's eye during the ST procedure is typically large enough to house the needle length and some other components so the width/thickness of the distal end of the probe 101 may be about 3 mm to about 30 mm or so, or more preferably about 4 mm to about 15 mm. In addition, the length (i.e. arc angle) of the curved end portion 103 of the probe 101 may be selected depending on the location of the eye that the needle is inserted at. The radius of curvature for the distal end portion 103 may be selected to be similar (i.e. approximately match or approximately the same as) to the curvature of the eye where the needle is inserted (e.g., sclera). Typically, the arc angle of the distal end portion of the probe may be in the range of about 0 degrees to 90 degrees, about 20 degrees to about 90 degrees, about 0 degrees to about 50 degrees or about 30 to about 40 degrees depending on the use case. Probes with different arc angles for the distal portion may be used when performing localization at different scleral locations. For example, an arc angle of about 0 to about 30 degrees may be used for anterior locations. In another example, an arc angle of about 15 to about 45 degrees may be used for mid periphery locations. In another example, an arc angle of about 30 to about 60 degrees or more may be used for more posterior locations. Similarly, probes with different lengths for the curved portion of the distal end portion (as defined by the arc angle of the distal end portion) may be used when performing localization at different scleral locations. It should be noted that the diagrams of the distal end of the probe 101 and 201 (see FIG. 2A) are not to scale and are provided as examples.

The distal end of the probe 101 may be used by a medical practitioner such as, for example, an ophthalmic surgeon or a vitreoretinal surgeon, hereafter referred to as a user, to maneuver around the curvature of the eye toward a location at the rear (i.e. posterior) of the eye, or any anterior or posterior location along the eye, depending on the ocular procedure being performed. For example, the posterior maneuvering may be to a location of a retinal break and to indent the sclera of the patient's eye during use. The placement of the distal end of the probe 101 on the posterior surface of the eye is such that it aligns with a desired location, such as the location of the retinal break in this example, which may be confirmed with the use of indirect ophthalmoscopy. For example, the user may be examining the posterior segment of the eye with a 28 D or 20 D lens and an indirect ophthalmoscope. One of the user's hands may be holding the lens and the other hand may be holding the probe 101 or 201, with the indirect ophthalmoscope being mounted on the user's head. The lens and/or indirect ophthalmoscope may also be considered to be a guidance tool. This then allows the user to determine (e.g., localize) a break location of the retinal tear or RRD of the patient's eye during use. In addition, the scleral depressor shape of the distal end of the probe 101 or 201 allows the user to access many portions of the posterior segment of the patient's eye as well as allow the user to apply pressure/depress the sclera of the patient's eye. This depression of the sclera allows the user to better visualize the retinal tear by looking inside the eye with indirect ophthalmoscopy. Once the depression is visualized and found to be well centered around the retinal break, the user may start injecting the treatment fluid using one of the techniques described herein. In the operating room it is also possible to visualize the inside of the eye with widefield viewing and direct illumination or illumination with a chandelier light source.

In at least one embodiment of the devices described herein, the distal end of any of the probes described herein is large enough to house (e.g., include space) for the extension/retraction of the needle and other elements that provide other functions. For example, one or more sensors may be located at the distal end of the probe. Alternatively, or in addition to the sensor(s), the distal end portion may have one or more openings or windows that perform various functions such as allowing a needle to extend from and be retracted into the distal end portion and allowing a light beam from a light source to be transmitted from the probe end. The light beam may be used to transilluminate through the sclera, to allow the user to know where exactly the needle entry point is, such as relative to a retinal tear location, for example. In one or more embodiments, the sensors, openings, windows, or other such features may be located along a surface of the side of the distal end portion and not at the tip of the distal end portion 102e (e.g., the end-face of the probe 101, 201 that is intersected by the longitudinal axis of the distal end portion of the probe).

In the example of treatment involving fluid injection, such as in retinal tear or RRD treatment, the fluid actuator 112 is fluidically coupled to the needle 106 and controllable (e.g., by the user) for causing a fluid, such as the treatment fluid, to move between the one or more probe conduits (e.g., from the injection conduit 104) through the needle conduit 106c and the SCS or other location of the eye. The fluid actuator 112 may be fluidically coupled to a treatment fluid source 116 via a tube 118, which may otherwise be referred to as tubing or a line, so that the fluid actuator 112 may cause the treatment fluid to go through tube 114 into the injection conduit 104. In other embodiments, the fluid actuator 112 may be contained within the body of the probe 102, and tube 114 may also be contained within the body of the probe 102 or may not be needed. The treatment fluid source 116 may be referred to more generally as a fluid source when fluid other than treatment fluids are injected. The treatment fluid may consist of a viscoelastic agent in the example of treating retinal detachment. However, more generally, in at least one embodiment described herein, a treatment fluid may include including any combination of any drug, gene therapy, stem cells, an extended release implant, a viscoelastic, a hydrogel, a gas or any other pharmacologic agent or material to be delivered into either the suprachoroidal space, the subretinal space, the sclera, the choroid or some other ocular location. The probe 101 may have a port 110 that serves to fluidically couple the tube 114 to the injection conduit 104. The fluid actuator 112 may include a pump that is used to apply an injection pressure to move the treatment fluid from the treatment fluid source 116 through the injection conduit 104 to the tip of the needle conduit 106c into the patient's eye. The injection pressure that is used may be predefined but may vary under certain circumstances. For example, the injection pressure may vary based on certain equipment factors such as the lumen size of the tubing and the size of the needle conduit 106c, as well as certain preferences that the user may have. For example, the injection pressure may vary from about 35 mm Hg to about 70 mm Hg although about 50 mmHg may be preferable. For example, the injection pressure used while the needle 106 is being advanced/extended into the patient's eye may be about 50 mm Hg so that the user can see the bleb forming in the SCS (i.e., the “SCS bleb”). A bleb refers to a blister, space, created between two tissue layers by a fluid, drug, or material. The formation of the bleb may vary based on the viscosity and other rheological properties of the fluid, drug, or material. Once the user sees the SCS bleb forming, the user may decide to increase or decrease the injection pressure to increase or decrease the rate of injection.

The treatment fluid source 116 may be a container (e.g., an injection container which may also be called an injection fluid container), a bag or a cartridge that includes the treatment fluid for provision to the probe 101 during use. For example, when the treatment fluid is in a cartridge, the cartridge may be removably slid into the main body 102 of the probe 101 and fluidically coupled with the conduit 104. Accordingly, in such cases, the fluid port may not be needed. In this case where the cartridge can be inserted in the main body of the probe, the cartridge may be a cylinder with an opening on one end that is covered by a membrane and there may be a spike in the device close to the proximal end of the conduit 104 that pierces the membrane to allow fluid from the cartridge to enter into the conduit 104 that is then in fluid communication with the needle conduit 106c. An actuator, such as a lever or dial (both not shown), may be used to add pressure to move the fluid through the needle conduit 106c. In at least one embodiment, the probe is pre-loaded with the treatment fluid or may be filled with the treatment fluid immediately prior to use by inserting a container, bag, or cartridge, or by filling a fluid chamber contained in the probe.

The fluid actuator 112 may be a motor in some cases that moves an object which applies a force to move the fluid into the eye. The force may be mechanical, or pneumatic and is applied at some point between the fluid source and the needle tip to eventually cause injection of the fluid into the patient's eye. The fluid actuator 112 may be coupled to a pedal and/or a switch, either in a wired or wireless fashion, where the pedal and/or switch are both configured to be controlled by the user that is using the device 100 to perform an ophthalmic procedure such as the ST method on a patient having an eye with a retinal tear or with RRD. While the user holds the probe 101 with one hand, the user may then use one of their feet to control the fluid actuator 112 via a pedal or the user may use the same hand if there is a switch on the probe 101 that is used to control the fluid actuator 112, while the user's other hand is holding a lens which is used to see into the patient's eye with an indirect ophthalmoscope. In an alternative embodiment, the fluid actuator 112 may alternatively be voice-activated so that the user can provide specific vocal commands to activate and deactivate the actuator. Upon activating the fluid actuator 112, the treatment fluid is provided from the treatment fluid source 116 through the tubes 118 and 114 to the injection conduit 104 and then to the needle conduit 106c for injection into the patient's eye.

Alternatively, when the target location is the SCS, the user may engage the fluid actuator 112 to start applying injection pressure when the position of the needle 106 is intrascleral (although there will be no or limited flow when the needle is intrascleral because the sclera will block the flow) and then slowly advance the needle 106 while continuing to apply injection pressure, and as soon as the needle tip enters the SCS, which provides less or no resistance to the flow of fluid from the needle, the fluid will start to flow and a choroidal buckle will start to form. By applying injection pressure as the needle is slowly advancing, it will result in choroidal bleb forming as soon as the needle enters the SCS, before the needle goes too deep into the eye (i.e. into the choroid). As soon as the needle enters the SCS, and a choroidal bleb forms, this can act as a visual notification and safeguard to the needle 106 entering into deeper structures such as the choroid or the subretinal space when treating retinal tears or RRD for example. The decrease in injection resistance and increase in fluid flow can also provide feedback to the user that the needle is in position and should not be advanced further. Injection resistance is the mechanical resistance to flow of fluid out of the needle and may be measured with a pressure sensor (pressure increases when trying to inject against a high resistance) or a flow sensor (flow stays low even when applying a pressure to inject if the resistance is high). The general relationship is Pressure=Flow*Resistance.

Accordingly, one or more sensors which can measure these values can be included in the device to provide data which may be referred to as location data. It should be noted that the bleb is not a leading bleb but a full bleb which is used to identify the space where the treatment fluid is to be injected since the flow is absent while needle 106 is in the sclera but present as soon as the needle enters the SCS. Accordingly, when the user is engaging the fluid actuator 112 so that it is active when the needle is in the SCS, the treatment fluid will be injected into the SCS of the patient's eye and when the user stops engaging the fluid actuator 112 so that it is inactive, the injection of the treatment fluid stops. This device setup allows the user to focus more on accurately holding the probe 101 in position during an ophthalmic procedure such as the ST procedure, while the switch/foot pedal/voice control allows for delivery of the treatment fluid in a controlled manner. For example, depending on the embodiment of the probe/device, voice commands may be provided for the automatic operation of the fluid actuator such as “inject now” or “stop injection”. The system may also have a confirmatory question such as “Are you certain you wish to start injection” to which the user answers “yes/no”. For the command “stop injection”, the fluid actuation may be disabled right away. Similar voice commands may be used for aspiration such as “Start aspiration now”, and “Stop aspiration”, for example.

The needle 106 has a needle position when extended from the body of the distal end of the probe, which in the embodiment of FIG. 1, is referred to as the injection position (in some embodiments, there may also be a drainage position where the needle is extended to a certain length to drain a certain region of the eye). The needle 106 also has a needle position when fully contained within the distal end of the probe, which is referred to as the retracted position. In some cases, it may be preferable for the needle to be in the retracted position while placing the probe in the desired location, to avoid scratching or otherwise injuring the eye, and only then extend the needle to the injection position. At the injection position, the end of needle 106 (i.e., needle tip) is adapted to extend about 0.3 mm to about 1.5 mm into the SCS of the patient for performing a first injection. This range in needle length is due to variations between patients and locations on the globe which may have different scleral thicknesses and require the tip of the needle 106 to be inserted at a larger or smaller depth. In at least one embodiment, the probe 101 may include a needle actuator 108 that is controllable (e.g. user adjustable) for adjusting the needle position. For example, if the user has to perform a second injection on the patient after performing the first injection, the user may use the needle actuator 108 to further extend the tip of the needle 106 so that it is inserted into a deeper location in the patient's eye such as about 1 mm to about 2 mm for a second injection into the SCS of the patient. The needle actuator 108 may also be used to move the needle from a retracted position to an extended position, or from an extended position to a retracted position. The needle actuator 108 may be engaged physically by the user or by using voice commands as described for the fluid actuator. In general, 0.8 mm may be used as a starting point, and if there is no choroidal elevation, the needle tip may be extended longer. However, in some locations, a needle extension of 0.8 mm may be too deep and the user may need to use a shorter initial needle extension for the needle 106. The depth may be assessed by the user under visualization with an indirect ophthalmoscope or in the operating room with wide-field viewing. It should be noted though that in some cases the scleral thickness will be less than 1 mm in which case the extension of the needle 106 will also be less than 1 mm.

Since the tip of the needle 106 may be extended for insertion into the patient's eye at different depths, the needle actuator 108 may include a needle position indicator (not shown) to visually indicate the insertion depth that is possible at a current needle position. For example, the needle actuator 108 may be a slider that is physically coupled to the needle 106 to move it to change the distance of the tip of the needle 106 relative to the distal end of the probe 101 from which the needle extends and the needle position indicator may include a line or tab on the slider and a numeric scale on the main body 102 of the probe indicating the length that the tip of the needle 106 can be inserted based on the current position of the needle 106. The slider may be manually controlled by the user to advance the needle 106 in small increments, such as 0.1 mm at a time, for example. Alternatively, a dial and a gear assembly or a lever may be used to allow the user to manually extend/retract the needle 106. In at least one embodiment, the needle actuator 108 may be motorized.

In an alternative embodiment, since it may be hard to see the needle length using the position indicator while the user is visualizing the inside of the patient's eye with a hand-held lens, a speaker may be used to produce audio output to inform the user the length of the needle 106 as the needle position is moved, so that the user knows the needle depth. For example, this might be performed by using a movement sensor that is coupled with the needle actuator 108 and the control unit 250 (e.g., see FIG. 2C) for sensing movements in needle position and generating needle movement data which is then processed by the control unit 250 which generates an audio signal that is provided as audio output via speaker 268. Alternatively, a series of ridges may be spaced apart at known distances, such as 1 mm for example, on the body 102 of the probe 101 and a bump on the needle actuator may be included so that each time the needle actuator 108 extends the tip of the needle 106 the bump on the needle actuator 108 may slide by one of the ridges on the body 102 and make a sound such as a click to inform the user that the needle tip has been extended by a distance equal to the spacing of the ridges on the body 102.

In at least one embodiment, the probe 101 may additionally include a sensor 107 disposed at a position in the injection conduit 104 or the needle conduit 106c to measure an injection resistance at about the distal end of the needle 106. In the example embodiment shown in FIG. 1, the sensor 107 is within a distal end of the conduit 106c. The sensor may be a pressure sensor and the injection resistance may be considered as being a back-pressure existing at the distal end of the needle conduit 106c and refers to the resistance encountered by the treatment fluid when it is injected into the eye. Alternatively, the sensor may be a flow sensor measuring the flow of fluid while an injection pressure is applied. Alternatively, the sensor may be a pressure sensor or a resistance sensor to measure the needle insertion resistance. The insertion resistance is the mechanical resistance to movement of the needle into the eye which may be measured with a mechanical force sensor or a pressure sensor. The sclera, SCS, choroid, and subretinal space of an eye all have unique insertion resistance values due to different densities/materials in these ocular locations. Accordingly, the sensor 107 may be used to measure an insertion resistance that may be used to approximate whether a tip of the needle 106 is in the sclera or the SCS or some other portion of the patient's eye. For example, in the case of an injection resistance sensor, the inventors have found that if the tip of the needle 106 is located in the sclera then there is a higher injection resistance but once the tip of the needle 106 is advanced into the SCS, the injection resistance reduces and a bleb forms at the tip of the needle 106 if injection has already been started by applying a sufficient amount of injection pressure. If the user starts the injection when the resistance is low then the treatment fluid propagates well into the injection site. Alternatively, the user may slowly apply an injection pressure while the needle tip is in the sclera and slowly advance the needle 106, and then once the insertion or injection resistance drops it indicates that the needle tip is in the SCS and a choroidal bleb will be visualized and the user can then increase the flow of the treatment fluid. For example, the actuator control may be implemented such that pressing harder on a switch or foot pedal connected to the fluid actuator 112 may increase the amount of treatment fluid (for example in a linear fashion, in which increased force leads to increased treatment fluid flow rate) that is injected or the amount of treatment fluid that is injected may be automated based on the measured injection or insertion resistance decreasing by at least about 50%. For example, this may be performed by the processor 270 of the control unit 250 (e.g., see FIG. 2C) upon receiving and processing the measured insertion values. Another alternative may be to initially create a choroidal bleb more anteriorly where it is easier to insert the needle 106 in the correct location, and then once a localized choroidal bleb is formed anteriorly, the needle length can be slightly increased, and a more substantial injection in the SCS can follow, such that the choroidal bleb propagates more posteriorly to the location of the retinal break.

The sensor 107 may also be any combination of an electrical impedance sensor, a mechanical resistance sensor and a flow sensor disposed at a position in the probe conduit or the needle conduit or on the external surface of the probe or needle to measure an insertion, injection, or electrical resistance at approximately a distal end of the needle where the resistance approximately indicates a position of a tip of the needle in the eye to determine whether the tip of the needle is in the sclera or SCS of the eye. The mechanical resistance sensor may be implemented using a pressure sensor or a force sensor. The electrical impedance sensor and the flow sensor are described in further detail below.

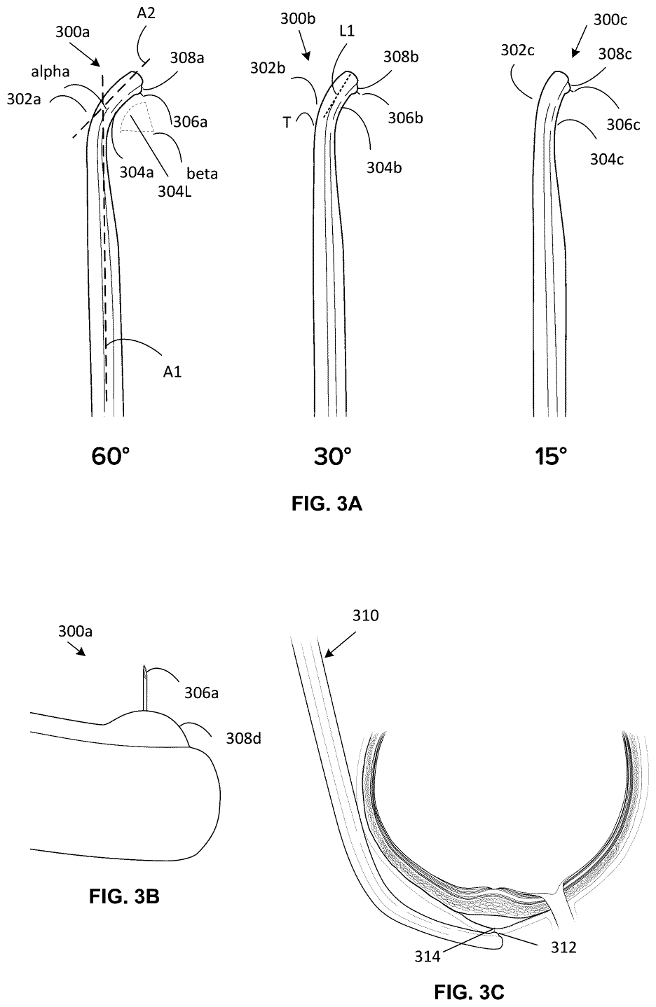

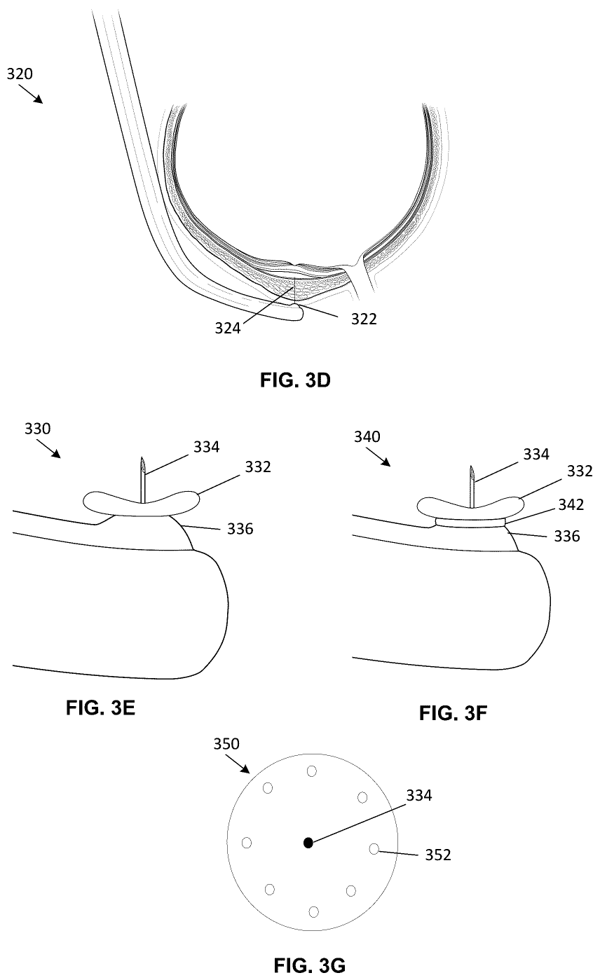

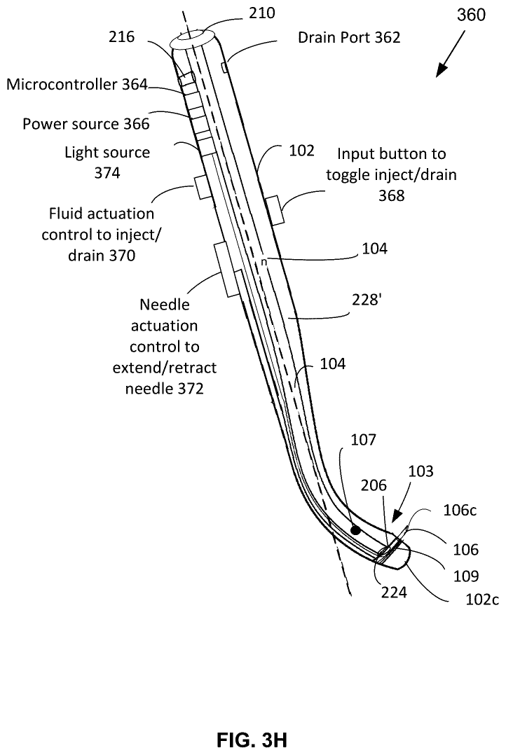

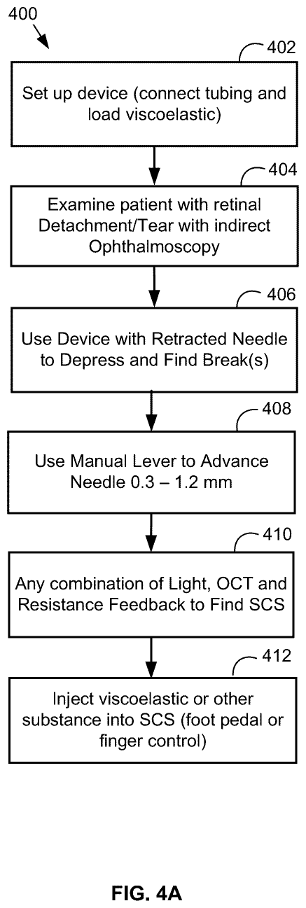

In at least one embodiment, the device 101 may have a processor and a display, such as an LED display, for obtaining and displaying the measured insertion, injection, or electrical resistance in order to provide a resistance feedback prompt to the user to inform them of when to begin the injection. In some embodiments, the display may be optional such as for the standalone device in FIG. 3H, for example. Alternatively, or in addition thereto, in at least one embodiment, the processor may be configured to automatically activate a fluid actuator to inject the treatment fluid when the resistance feedback indicates that the needle tip is in the correct location for injection of the treatment fluid. In at least one embodiment, the measured resistance values may be compared to a resistance threshold and once the measured resistance value is lower than the resistance threshold the resistance feedback prompt may be provided to the user through a visual display or through audio and/or a fluid actuator may be activated to automatically begin injection of the treatment fluid. Alternatively, in at least one embodiment, the measured resistance values may be analyzed to determine a rate of change and when the magnitude of the rate of change is larger than a resistance change threshold the resistance feedback prompt may be provided to the user visually and audibly and/or the injection automatically started. Such threshold values can be determined experimentally and may depend on any combination of patient age, patient sex and whether the patient has an eye condition that leads to thicker or thinner sclera.

As described above, in at least one embodiment, the probe 101 and/or 201, may use electrical impedance to detect whether the needle tip is in the sclera or the SCS since the electrical impedance of different tissue types may be different. For example, the resistance sensor may be an electrical impedance sensor which may include a measurement electrode that may be located at or near the tip of the needle 106 and a reference electrode also located near the tip of the needle 106 or in another portion of the needle 106 or the probe 101, 201 and connected to ground. As the measurement electrode comes into contact with the scleral tissue when the needle tip is extended into the patient's eye, impedance measurements are taken which will be approximately at a first value that may be referred to as the scleral impedance. As the needle tip is further extended and enters the SCS, impedance measurements will continue to be taken and will be approximately at a second level that may be referred to as the SCS impedance. The scleral impedance is different than the SCS impedance. Accordingly, impedance values can be measured as the needle tip is extended into the patient's eye and when the measured impedance value changes from the scleral impedance value to the SCS impedance value, a signal can be provided to the user to notify them to start injecting the treatment fluid and/or the fluid injection may be automatically started as described previously.

It should be noted that the depth of the tip of the needle 106 when the needle 106 is in the SCS may vary from patient to patient and from one location to another location in a given patient's eye. Accordingly, in at least one embodiment, the tip of the needle 106 may be extended to an initial length for SCS injection but may then slowly be extended little by little (e.g., incrementally) and the injection may occur little by little (e.g., incrementally) until the measured insertion resistance indicates proper placement of the tip of the needle 106. For example, it may be that once the distal end 103 of the probe 101 is in the region of the retinal tear or break (which is being observed with indirect ophthalmoscopy based on the indentation created by the distal end of the probe or with widefield viewing), the needle 106 may be controlled to penetrate the sclera slowly, such as in an incremental manner, which may be done in a motorized manner, such as by using a stepper motor for example, and as the tip of the needle 106 is extending, the user can see the needle tip indenting the posterior sclera or visualize the needle as it pierces the sclera (with the assistance of a light shining through the lumen of the needle) of the eye and the user can confirm visually that the needle 106 is not too deep, and the user can keep extending the needle 106 until a set length of say about 0.3 mm, about 0.9 mm, about 1 mm, about 1.2 mm or whatever depth the user chooses and/or the user can also use visual cues to limit the needle depth penetration. For example, a guidance tool (described below) may be used to view the action of the needle tip, including to see the indent of the needle and to see when the needle has passed the sclera and is at the SCS and in the case that injection pressure is being applied see the fluid start to be injected and can stop the needle extension so that it does not penetrate deeper structures. These approaches provide safeguards against too deep penetration. The gradual extension of the needle 106 may be implemented using a processor-controlled stepper motor that is coupled to a slider, a rotating dial or the like for sliding/extending the tip of the needle 106 away from the distal end of the probe 101 in an incremental manner and also retracting the needle 106 within the distal end of the probe tip. There may be a switch or foot pedal that is user controlled and is communicatively coupled to the stepper motor to provide the user with finer control of incremental extension and retraction of the needle 106 during use. The switch or foot pedal mechanism may be the same one that is used for automatically injecting the treatment fluid such that there are two switches on the probe 101, two buttons on the probe 101 or two portions of the foot pedal and the user may engage one of the switches or a first part of the pedal to move the needle and then another switch or a second part of the pedal to start the injection of the treatment fluid or to start aspiration (e.g., see below). Alternatively, or in addition thereto, in at least one embodiment, voice control may be used to control extension/retraction of the needle. These mechanisms may also apply to other probes described herein such as probe 201.

For example, during use, the user is indenting on the globe with the distal end of the probe 101 or 201 while the needle is retracted, and once the correct location on the globe is identified, the user activates the motor control and the needle 106 starts to extend slowly. As the needle 106 is being extended the actuator for injecting the treatment fluid is also activated, however, the treatment fluid will not come out of the needle tip when the needle tip is in the sclera (due to not using a large enough injection pressure to overcome the injection resistance in the sclera), until the needle tip enters the SCS where a bleb will automatically form. Once the bleb has started to form, the user can increase the flow rate to complete the delivery or continue with the same flow rate. Alternatively, in at least one embodiment, the user may control the device to reduce the flow rate so that the treatment fluid is delivered more slowly to make the injection more controlled. In at least one embodiment, the probe 101 or 201 may have a flow sensor to detect when the flow of treatment fluid out of the needle tip begins. When the flow is detected, if a motor is being used to extend the needle tip, it may be turned off to stop advancing the needle tip. At this point the needle may be locked in place and the treatment fluid may be continued to be injected until a sufficient amount has been injected (as described herein). Also, as the fluid flows into the SCS, the intraocular pressure may rise.

In at least one embodiment, two separate input mechanisms can be used including one (e.g., first) mechanism/input device for controlling the fluid actuator and another (e.g., second) mechanism/input device for controlling the needle actuator. The first and second mechanisms/input devices may each be a dial, a slider or button.

In an alternative embodiment, any of the probes described herein include a pressure sensor, which may be located near the distal end of the probe or another suitable location and coupled with the injection conduit 104, that can be used to obtain intraocular pressure measurement data. The intraocular pressure data may be processed by a processor to alert the user when the intraocular pressure has reached an intraocular pressure limit where it is advisable to stop further injection of the treatment fluid to avoid any damage to the eye. This pressure limit may be predefined and it may be adjusted based on the type of ocular procedure being performed and the health of the eye receiving the procedure. For example, the intraocular pressure threshold may be about 40 mm Hg, about 60 mm Hg, or about 80 mm Hg. The intraocular pressure measurement may be based on scleral parameters or other ocular tissue parameters sensed by the device. The device may then indicate to the user when the intraocular pressure has reached 40 mm Hg, 60 mm Hg, or 80 mm Hg. In the inventors' experience, the pressure can rise with the suprachoroidal injection such that the central retinal artery is occluded or pulsatile. In these cases, based on the inventors' experience, the user may perform an anterior chamber paracentesis to reduce the intraocular pressure. The removal of fluid from the anterior chamber can be done slowly and in small increments to avoid bleeding in the posterior segment at the site of needle penetration. In some cases, to reduce the intraocular pressure subretinal fluid may be drained slowly or some of the treatment fluid may be removed with the device.

In an alternative embodiment, robotics may be used where sensor feedback is used by a robot to automatically advance the needle to the desired location. The robot may receive sensor data such as injection or insertion resistance and use this data to slowly advance the needle. Once the desired sensor values are reached, the robot may start the injection until a desired volume has been injected. The robot may also use sensor data such as intraocular pressure and/or injection resistance to determine when to not inject any further. The surgeon can override the robot at any time to modify needle location or to control treatment fluid injection.

In at least one embodiment, the device 100 may include a guidance tool which may provide guidance based on electrical, mechanical, optical, acoustic, force, pressure, flow, image, or tomographic data. For example, in at least one embodiment, the guidance light tool includes a light source 122 that generates a guidance light beam. The guidance tool is optically coupled to the distal end of the probe 102, via an optical fiber 120 that is coupled to the port 110. The probe 101 may have an internal optical fiber 109 with a proximal end that is coupled to the port 110 and a distal end located at the distal end of the probe 101 to transmit light from the guidance tool to the distal end of the probe 101 (e.g., the optical fiber 109 may be passed into or be adjacent to the needle conduit 106c) so that light is emitted at the distal end of the probe 101 allowing the user to see the light while examining the patient's eye with indirect ophthalmoscopy, thereby allowing the user to know if the needle 106 is at an intrascleral position, in the SCS, or deeper into the subretinal space. The optical fiber 109 is thin enough that it will not interfere with the injection of the treatment fluid. The light provided by the optical fiber 109 may allow for a portion of the eye in the vicinity of the tip of the needle 106, such as the sclera or choroid, to be visualized so that the user can more precisely locate (i.e., align) the tip of the needle 106 at the proper location to inject the treatment fluid. For example, such an optical fiber 109 when shining light (e.g., illuminating the tip of the needle 106 during use) may allow the user to visualize the needle tip internally when the needle 106 is in the suprachoroidal space (or if the needle tip is too superficial in the sclera or too deep in the subretinal space). If the optical fiber 109 is thin, the light intensity may have to be increased to improve visualization. Alternatively, if the injection conduit 104 is made using material that has sufficient internal light reflection properties, then the injection conduit 104 may be used instead of the optical fiber 109. The optical fiber 109 may be replaced with a conduit for a different guidance modality, such as when a non-light guidance tool is used such as an ultrasound imaging device, for example.

When performing visualization using the optical fiber 109, there may be a change in the light intensity and/or light color when the needle tip extends from the sclera into the SCS (and into the choroid, subretinal space and/or other portion of the eye) which provides a visual cue to the user that the needle tip is in the SCS (or in the sclera, the choroid, the subretinal space, or some other portion of the eye). The light intensity may then change further as the treatment fluid is injected. In at least one embodiment, the light that is transmitted by the optical fiber 120 may have a color (other than white) to help the user with identification of the light during visualization and the light transmitted by the optical fiber can be referred to as a guide beam. For example, the color may be blue, green or red. In either case of white light or colored light, when the needle 106 is in the sclera there will be an internal dull (e.g., attenuated) colored light seen as the colored light transilluminates the globe/location. As the needle 106 advances through the sclera, the colored light will increase in intensity or be visible for the first time which can be recognized by the user to know the needle has passed the sclera and is in the suprachoroidal space. The sclera is opaque tissue and tightly packed collagen fibers. As such when the light is shone from outside the sclera or intrascleral, less of the light will be transmitted. Also, the appearance of the light may look different when it is intrascleral compared to in the SCS, because the color of the light may be modified by the tissue it is passing through. For example, when the light is directly under the choroid it can change in color compared to when it is intrascleral. This is because light at different wavelengths may be absorbed differently by the sclera and the SCS (and the choroid and the retina), and so these different light wavelengths may be useful in differentiating between the scleral and SCS (and other location) positioning of the needle tip, where the wavelength may correspond to that of a red, blue, or green light as examples. The change in light intensity and/or color will be visible to the user who is examining the patient's eye with indirect ophthalmoscopy and a condensing lens. Once the colored light is more clearly seen, which may happen when the needle 106 has passed through the sclera into the SCS, the injection can be initiated by the user.

In at least one embodiment, the spot size of the guidance light beam that is used may be in the range of about 50 microns to about 500 microns and the intensity of the light beam may be selected so that changes in the light property are visible to the human eye and/or detectable by a light sensor. In at least one embodiment, an optical fiber transmitting the guidance light beam may be provided in addition to an optical fiber that transmits white light for viewing/imaging. The white light beam may be larger and may be provided by an optical fiber that is coupled to the distal end of the probe 101, 201.

It should be noted that there may also be a change in light intensity when the needle tip is directed perpendicularly into sclera (e.g., the light intensity may be the highest in this case). Alternatively, if the light beam consists of polarized light, a light polarization sensitive sensor may be used to ensure that the needle tip is at a perpendicular orientation with respect to the needle insertion made in the sclera.

Accordingly, in at least one embodiment, any changes in light property of a light beam that is transmitted via the probe into the eye, where the light property may be intensity, wavelength (e.g., color), and/or polarization, may be detected by sensing any reflected light using a light sensor to obtain reflected light data and using a processor to analyze the reflected light data. The change in light property may be communicated to the user via the processor. For example, a sensor may be used to sense back scattered or reflected light of a certain wavelength, to know when the needle has passed through the sclera. The reflectance of the light back to the sensor may be different when the needle is in in the sclera as opposed to when it has passed the sclera and entered the SCS.

Alternatively, in at least one embodiment, the guidance tool may be an imaging and/or measurement device 122 such as an Optical Coherence Tomography (OCT) device. The guidance tool is optically coupled to the distal end of the probe 102, via an optical fiber 120 that is coupled to the port 110. The probe 101 may have an internal optical fiber 109 that is coupled to the port 110 or the injection conduit 104 may be used to: (a) transmit light from the OCT device to the distal end of the probe 101 so that light is emitted from the distal end of the probe 101 into the area of the patient's eye, such as the sclera or choroid, where the tip of the needle 106 is located; and (b) transmit reflected light from this area of the patient's eye to the OCT device 122 which may then generate and display OCT images based on the reflected light. The user may view the OCT images to more precisely locate (i.e., align) the tip of the needle 106 at the proper location to inject the treatment fluid. In at least one embodiment, the OCT image may be used to ensure perpendicular alignment by measuring the thickness of the sclera and adjusting the probe angle to reduce (i.e., preferably minimize) the scleral thickness, which indicates a perpendicular crossing (i.e., the shortest path) rather than an angled approach for the needle insertion.

It should be noted that in at least one embodiment, other light guidance tools may be used such as an Optical Coherence Elastography (OCE) device, an endoscope imaging device, a light intensity sensing device, a light scattering sensing device, a light wavelength sensing device, or a light polarization sensing device. In the case of an endoscope, a light camera is used so that the user can view on a monitor where the needle tip is. This may also allow the user to see the scleral fibers and know when the needle tip is in the SCS for example.

It should be noted that for the various optical imaging devices described herein, the implementation may involve using flexible optical fibers (or other ‘optical conduits’) that bend to come out of the side of the probe distal end or in some cases a front-facing imaging tool may be used along with a 45 degree mirror such that the tool can generate images with a field of view orientated perpendicular to the longitudinal axis of the distal end (i.e. towards the eye) where the needle will penetrate during use, rather than directed toward the tip of the distal end.

It should also be noted that the various optical imaging devices and one or more of the sensors described herein within the probe may be analyzed to determine location data indicating where the needle tip of the probe is located during use. The combination of imaging devices and one or more sensors may provide an assessment of whether the needle is intrascleral or if the needle has fully passed through the sclera and is in the suprachoroidal space. For example, the needle depth penetration may be determined by matching the optical, mechanical, electrical, or other outputs of the imaging devices and the sensors with the different values of properties of different types of eye tissue. The needle depth penetration may also be determined by counting the number of times the output measurements change, with each change indicating that the probe has passed from one tissue type to another, and correlating this with the known tissue layer anatomy of the eye. In general, the needle depth penetration may be determined according to the location of the imaging device and/or sensors, with the location of the needle tip calculated relative to that reference location based on known (calibrated) physical configuration of the probe elements. The needle depth penetration data (i.e., location data) may be displayed by the control unit 250 or otherwise communicated to the user such as through audio feedback.

In yet another alternative, in at least one embodiment, the guidance tool may be an imaging device 122 such as an ultrasound (US) imaging device. The guidance tool is coupled to the distal end of the probe 102 via an electrical wire or cable 120 that is coupled to the port 110. The probe 101 may have an internal electrical wire that is coupled to the port 110 and an US transducer disposed near the distal end 103 of the probe 101. The US transducer can transmit sound waves from the distal end of the probe 101 into the area of the patient's eye, such as the sclera or choroid, where the tip of the needle 106 is located; and (b) the reflected US waves from this area of the patient's eye are then received by the US transducer which converts corresponding electric signals that are sent to the US imaging device 122 which may then generate and display US images based on the reflected US waves. The user may view the US images to more precisely locate (i.e., align) the tip of the needle 106 at the proper location to inject the treatment fluid. In addition, in at least one embodiment, the US data/US images may be used to align the needle so that it has an approximate perpendicular entry into the sclera.

The treatment fluid obtained from the treatment fluid source 116 may be a viscoelastic agent such as hyaluronic acid, for example, which may have an active period (last for) of a few weeks (2-8 weeks). For example, sodium hyaluronate 2.3 may be used which may have an active period of about 2-3 weeks. However, longer lasting treatment fluids may be preferred such as “cross-linked” hyaluronic acid or otherwise modified hyaluronic acid. For instance, Restylane may be used which may be active for several months. Alternatively, different compounds that are absorbable and are inert/non-inflammatory may be used for the treatment fluid. For example, sodium hyaluronate 1%-2.3%, Restylane, or hydrogel spacers (dissolvable or non-dissolvable) may be used.

In alternative embodiments, the treatment fluid may include an inert gas or air.

It should be noted that in at least one alternative embodiment of the device 100, the fluid actuator 112 may be provided by the plunger of a syringe and the treatment fluid source may be contained in the barrel/chamber of the syringe. In this case a person assisting the user may actuate the plunger to do the injection.

In another alternative embodiment, the injection conduit 104 may be preloaded with the treatment fluid, or the treatment fluid source may be incorporated into the injection conduit, such that the device does not need to be coupled to an external treatment fluid source. In such cases, the fluid actuator 112 may still be used to apply an injection pressure in a similar manner as described previously so that the user is able to have one hand dedicated to the handling of the probe 101.

Referring now to FIG. 2A, shown therein is a diagram of another example embodiment of the probe 201 of an ocular treatment device 200 for treating retinal tear or RRD, or for delivering a therapeutic fluid to the eye, in accordance with the teachings herein. The probe 201 has some similarities to the probe 101 and thus has similarly numbered components that operate in a similar fashion and are not discussed in detail as they have already been discussed in relation to probe 101. However, the probe 201 also has additional components that allow a user to aspirate fluid (actively or passively) from the patient's eye, such as suprachoroidal fluid or hemorrhage or sub-retinal fluid (SRF) or subretinal hemorrhage or some of the injected fluid when too much fluid is injected, which may need to be done on a patient-by-patient basis. For example, the probe 201 includes a dual conduit design including a drainage conduit for draining fluid from a region of the eye, such as subretinal fluid for example, and an injection conduit for providing a treatment fluid for injection into the SCS of the patient's eye for treating a RRD or retinal tear (or into a different location for treating a different condition) as was described previously for probe 101. The drainage conduit may be fluidically coupled to a drainage tube that is within or external to the probe and stores the drained fluid.

In an alternative embodiment, a probe is provided that may use a single conduit that can function as both an injection conduit and a drainage conduit at different times as long as any drained fluid is removed from the probe and does not interact with any fluid that is injected. In some cases, drainage may be performed first and then injection, or in some cases fluid injection may occur first and then drainage may be performed in the same location or elsewhere. In such embodiments, a valve or gate may be used to fluidically couple the single conduit to a fluid source or a drainage container when injection and drainage are being performed, respectively. Accordingly, in at least one embodiment, an injection fluid container and/or a drainage fluid container is coupled to the one or more probe conduits.

FIG. 2A shows that the probe 201 has a dual cylinder design with an inner cylinder being surrounded by an outer cylinder and one of the cylinders acting as an injection conduit with the other cylinder acting as a drainage conduit. Accordingly, the probe 201 includes a cylinder that acts as the injection conduit 104 and may be pre-loaded with the treatment fluid in a fluid cartridge that may be coupled to the injection conduit 104 as explained for probe 101. In at least one embodiment, the probe 201 may include a plunger 211 that has a shaft 213 that is slidably received within a port 212 of the injection conduit 104. The plunger 211 has a thumb rest 215 that the user may push when injecting the treatment fluid. The user may then push on the thumb rest 215 to inject the treatment fluid.

Alternatively, the probe 201 may not include the plunger 211 but rather uses similar components to those used with probe 101 in device 100 (e.g., fluid actuator 112, pump(s) (not shown in FIG. 1 but an example are pumps 280 in FIG. 2C) as well as tubes 114 and 118) for injection of the treatment fluid. Alternatively, in at least one embodiment, a hardware configuration such as the control unit 250 shown in FIGS. 2B and 2C may be used with any of the probes described herein for actuating the injection of the treatment fluid into the patient's eye, in which case the control unit is separate from (e.g., not contained in or housed with) the probe. In such designs, coupling for providing the treatment fluid to the injection conduit 104 may be through port 212 or alternatively the port 212 may not exist and the end of the injection conduit 104 is sealed while a side injection port 216 is used to provide treatment fluid to the injection conduit 104. Accordingly, the port 216 may be optional in some embodiments or the port 212 may be optional in other embodiments. However, there may be embodiments in which the control unit is housed within the probe (e.g., see the standalone probe/device in FIG. 3H).

In at least one embodiment, the probe 101 may include one or more resistance sensors 107 that are located on the underside of a stopper 214 at the end of the distal end of the plunger 212 adjacent the treatment fluid. Alternatively, in some embodiments, such as those that do not include the plunger 212, the resistance sensor(s) 107 may be located in the needle conduit 106c such as near the distal end of the needle conduit 106 similar to what was described for probe 101. The resistance sensor(s) 107 operates as was described for probe 101.

The outer cylinder may act as the drainage conduit 228 for draining fluid from a portion of the patient's eye, such as subretinal fluid, for example. The needle 106 has a needle position that is adjustable between a drainage position for fluidically coupling the drainage conduit 228 to the needle conduit 106c for draining fluid from the subretinal space of the eye and an injection position for fluidically coupling the injection conduit to the needle conduit for injecting the treatment fluid into the SCS of the eye. When the needle position is in the injection position (this position is what is shown in FIG. 2A), a circumferential aperture 224 (e.g., pore) is located to coincide with the injection conduit 228. When the needle position is in the drainage position, the needle 106 is advanced so that the tip of the needle 106 extends further away from/beyond the distal end 103 of the probe 201 which advances the position of the circumferential aperture 224 so that it is located to coincide with the drainage conduit 228. Accordingly, the aperture 224 and elements that move the needle 106 may act as a coupling that is adjustable between fluidically coupling the drainage conduit to the needle conduit and fluidically coupling the injection conduit to the needle conduit.

The position of the needle 106 is controlled by the needle actuator 108 as described previously for probe 101. In the embodiment of FIG. 2A, the needle actuator 108 is connected to a slider mechanism 220, or other suitable mechanism, to advance or retract the tip/end portion of the needle 106. Alternatively, a rotary dial may be used instead of the slider 220. In either case a gear assembly may be used so that a larger movement of the slider or dial is translated into a smaller motion of the extendable needle 106 to allow the user to more finely control the movement of the needle 106, or vice versa. The needle 106 is also located within a sleeve 206 that constrains the motion of the needle 106 to be linear as it extends and retracts outside of the distal end of the probe 201. For example, when the needle position is in the injection position, the tip of the needle 106 may extend between about 0.3 mm to about 1.5 mm for the first SCS injection or about 1 mm to about 2 mm for the second SCS injection (as explained previously). For example, the needle actuator 108 may be implemented to move in incremental steps that correspond to incremental changes in the depth of the tip of the needle 106 into the patient's eye, such as, but not limited to, incremental steps corresponding to 0.9 mm, 1 mm, 1.1 mm, 1.2 mm, 1.3 mm, 1.4 mm and 1.5 mm, or other suitable distances, for example.

However, for probe 201, the needle position may be changed to the draining position in which case the tip of the needle 106 may extend about 1.5 mm to about 3 mm away from/beyond the distal end of the probe 201 in order to reach a subretinal location of the eye of the patient. The needle depth used for drainage depends on the drainage location (e.g., which can change depending on thicknesses of certain ocular layers which can vary for different people e.g., such as the sclera). However, if the retina is more bullously detached, the tip of the needle 106 may be advanced slightly more whereas if the retina has a shallow detachment then a shorter needle length may be used for drainage. The needle length may be changed in fine increments such as, but not limited to, 0.1 mm for example so that it can be extended or retracted slowly as described previously. For example, the needle tip may be advanced slowly until it passes through the retinal pigment epithelium and into the subretinal space and as the drainage comes to an end and the retinal detachment height is reduced, the needle 106 can be retracted to avoid touching the retina. The fluid drainage may be passive (in which fluid at a higher pressure in the eye drains naturally into a lower pressure drainage conduit) or it may be active in which case suction is used. For example, the fluid actuator 112 and a pump may be coupled to the drainage conduit 228 via port 210 and operated in reverse so instead of applying an injection pressure, the fluid actuator 112 applies a drainage pressure that is negative (e.g., suction). In at least one embodiment, the control unit 250 may instead be used to provide the active drainage. Accordingly, in various embodiment, there is a pump fluidically coupled to the one or more probe conduits that is controllable to create an injection pressure when the fluid is injected into the eye or a drainage pressure when the fluid is drained from the eye.

The inventors have also discovered that to achieve good results in repairing an eye having retinal detachment or a retinal tear, drainage of fluid from the eye may be needed in some cases. The fluid drainage may be for fluid that is in the subretinal space (approx. 1.5-3 mm depth). After that the needle position may be changed to the injection position where the needle 106 is retracted to a shorter length (i.e., a shallower depth into the patient's eye) to then inject the treatment fluid into the SCS. This may be performed as a two-step procedure where there may be different locations for eye fluid drainage (from subretinal space) and the treatment fluid injection (in the SCS) but in some cases this drainage and injection might be at the same location where the retinal tear is located. However, in some cases, it is not desirable to drain near a larger retinal tear because then there is a chance of draining vitreous fluid rather than subretinal fluid. In cases where there are larger breaks, the user may choose another location to drain the subretinal fluid, particularly where the retina is more bullously detached.

The probe 201 includes a port 222 for receiving a portion of a guidance tool to aid the user in properly positioning the tip of the needle 106 when the user is performing draining or injection. The guidance tool is similar to those described above for device 100. Accordingly, when the guidance tool includes a light source, the port 222 may receive an optical fiber that may be used for coupling to an internal optical fiber 109 for illuminating the tip of the needle 106 during use. Alternatively, the port 222 may be used to connect with an OCT imaging system (or any of the other imaging systems described previously) when the guidance tool includes the OCT imaging system (or any of the other aforementioned imaging systems). Alternatively, the guidance tool may be an ultrasound imaging device in which case port 222 may be used to couple an electrical wire from the US imaging device to an internal electrical wire that is connected to an ultrasonic transducer located at the distal end of the probe 201 as was explained for the probe 101.

Referring now to FIGS. 2B and 2C, shown therein is a front view of an example embodiment a control unit 250 and a block diagram of the electrical components of the control unit 250, respectively, where the control unit 250 may be used with any of the embodiments of the probes described in accordance with the teachings herein to help the user in performing the ST technique or another ophthalmic procedure/treatment. For example, the control unit 250 may be used with the probe 201 together forming an embodiment of an ocular treatment device. Alternatively, the control unit 250 may be used with the probe 101 when the control unit 250 is operating in injection mode only. The control unit 250 may be used to perform certain functions such as, but not limited to, any combination of controlling drainage, controlling injection, indicating certain operating parameters to the user, displaying visual guidance to the user using the guidance tool and providing feedback prompts to the user. In at least one embodiment, the control unit 250 may be used to control needle insertion/retraction by processing any combination of sensor signals, and imaging data to extend the needle to a desired injection location and retract the needle as needed during a procedure. The control unit 250 is connected to an actuator control input 262 such as a pedal, switch, slider, rotational dial and the like that the user may use to perform injection or drainage while leaving at least one of their hands free to hold the probe 201 at the precise location. It should be noted that the control unit 250 may be used with the probe 101 in which case the components shown in FIGS. 2B and 2C related to drainage are not needed when the probe 101 does not perform drainage. It should be noted that the components and position of the components on the control unit 250 is provided as an example only and may be different in other embodiments.

The control unit 250 includes an on/off button 252, an injection operation button 254 and an aspiration operation button 256. In other embodiments, these buttons may be implemented using one switch with an injection position and a drainage position, separate switches for selecting the injection mode and the drainage mode or other suitable input/control elements. The user may push the on/off button 252 to turn the control unit 250 from off to on or on to off. The user may use the injection operation button 254 to switch the control unit 250 into injection operation mode in order to inject the treatment fluid into the patient's eye via the probe 201 or 101. The user may use the aspiration operation button 256 to switch the control unit 250 into aspiration operation mode in order to drain fluid from the patient's eye via the probe 201.

The control unit 250 also includes an injection port 258 to receive external injection tubing that is connected to the injection conduit 104 of the probe 101 or 201. The interior of the injection port 258 is connected to an internal injection conduit (e.g., internal tubing) which is connected to a pump 280 that is used to generate an injection pressure during injection. The pump is fluidically coupled to a treatment fluid source to send the treatment fluid to the injection port 258 via the internal injection conduit. The control unit 250 also includes a drainage port 260 to receive external aspiration tubing that is connected to the drainage conduit 228 of the probe 201. The interior of the port 260 is connected to an internal drainage conduit (e.g., internal draining tubing) which is connected to the same pump 280 or a different pump that is used to generate a suction pressure during drainage. The pump 280 is operated in response to the user engaging the actuator control input 262 in the on position. The pump 280 is fluidically connected to a drainage container (e.g., drainage fluid container) which receives the drained fluid during use.

Engaging the actuator control input 262 will send an actuator control signal that is received by a processor 270 of the control unit 250 which in turn generates a pump control signal to operate the pump 280 according to injection mode or aspiration mode. In injection mode, the processor 270 is configured to send a pump control signal to the pump 280 to create an injection pressure at a predefined injection pressure level to move the treatment fluid from the treatment fluid source to the SCS of the patient's eye. For example, the predefined injection pressure may be in the range of about 20 mm Hg to about 80 mm Hg such as 50 mm Hg, for example. In drainage mode, the processor 270 is configured to send a pump control signal to the pump 280 to create an aspiration pressure at a predefined aspiration pressure level to move the drainage fluid from the patient's eye to the drainage container. For example, the aspiration pressure may be preset to be in the range of about 0 to 700 mm Hg or from about 100 to about 650 mmHg.

The control unit 250 may include a display 264 for displaying/showing various information to the user. For example, the display 264 may be used to show the insertion resistance that is measured during operation, the injection pressure that is used during injection mode, and the aspiration pressure that is used during aspiration mode. Other measurements that are made during operation, depending on the embodiment of the probe as described herein, may be displayed such as light intensity, light wavelength and/or light polarization of any reflected light from the eye into the probe that is measured, for example. In the case of the measured injection resistance, there may be an electrical wire/cable connecting the resistance sensor in the probe 101, 201 with an interface in the control unit 250 that is accessible by the processor 270. Alternatively, in at least one embodiment the connection between the probe and the control unit 250 may be wireless. In at least one embodiment, the display 264 may be a touch sensitive screen that may be used by the user to operate the control unit 250 in which case the injection operation button 254 and the aspiration operation button 256 may be omitted as the user may select these modes through touching the display 264.

In at least one embodiment, the control unit 250 may include a guidance tool port 266 when a guidance tool is used such as a light guide, OCT imaging and/or US imaging. In the case of a light guide, the port 266 may be used to receive an external optical fiber that is connected to the probe 101 or 201 and internally the port 266 is connected to an internal light source. In the case of OCT imaging, or the other light imaging devices described previously, the port 266 may be used to receive an external optical fiber that is connected to the probe 101 or 201 and internally the port 266 is connected to an OCT device (or other light imaging device as described previously) which may be internal or external to the control unit 250. In the case of US imaging, the port 266 may be used to receive an electrical cable that is ultimately connected an US transducer in the probe 101 or 201 and internally the port 266 is connected to an US imaging device which may be internal or external to the control unit 250. When the guidance tool is an OCT device or US imaging device and it is included with the control unit 250, the resulting OCT or US images may be output on the display 264.

When the guidance tool is a light guide and the display 264 is a touch sensitive display, the user may be able to adjust an intensity or wavelength of the light generated by the light source. Alternatively, a physical slider control (not shown) may be used for the same purpose in embodiments where the display 264 is not touch sensitive. The light source may be an LED that generates white light and an LED driver may be used to control the intensity of the generated white light beam. The light intensity may be controlled via calibration settings and/or via user control during use. In at least one embodiment, the light source may include an LED that can generate a colored light beam as explained previously. In at least one embodiment, the light source may include two LEDs with two LED drivers where one of the LEDs generates white light and the other LED generates a colored light. Optics may be used so that the colored light beam is smaller and disposed within the white light beam.

In at least one embodiment, the control unit 250 may include a speaker 268 which may be used to output speech indicating one or more operational parameters and/or the operational mode of the control unit 250. Accordingly, when the user selects injection mode or aspiration mode, a speech audio may be output through the speaker 268 indicating that the control unit 250 is operating in injection mode or aspiration mode, respectively. Furthermore, any of the measured injection resistance, the injection pressure and the aspiration pressure may be indicated in a speech audio that is output though the speaker 268. In the case of outputting the measured insertion resistance, this may be done as the user is inserting the needle 106 and the measured insertion resistance changes by a certain amount, such as between 10% to 20% for example. Alternatively, other sounds, such as tones or beeps, may be communicated to the user depending on the situation during operation such as when the needle tip is penetrating too deep or arriving at its intended destination based on any of the sensing techniques described herein.

Referring again to FIG. 2C, shown therein is a block diagram of an example embodiment of the components of the control unit 250 of FIG. 2B. The control unit 250 includes the processor 270, a memory unit 272, a user interface 274, a device interface 276, one or more sensor(s) 278, the display 264, one or more of the pump(s) 280, the speaker 268, an optional light source 282, an optional OCT device 284, an optional US device 286 and power supply unit 287. The memory unit 272 includes random access memory (“RAM”) and non-volatile storage that is configured to store program/software instructions for an operating system 288, programs 290, a control application 292, an I/O module 294 and files 296. The components and organization of the components shown in FIG. 2C are one example and may differ in other embodiments. The power supply unit 287 provides power that is supplied to various components of the control unit 250.

The processor 270 controls the operation of the control unit 250 and can include any suitable processor, controller or digital signal processor that can provide sufficient processing power depending on the configuration, purposes and requirements of the control unit 250 as is known by those skilled in the art. For example, the processor 270 may include a high-performance general processor. In alternative embodiments, the processor 270 may include more than one processor with each processor being configured to perform different dedicated tasks. In alternative embodiments, specialized hardware can be used to provide some of the functions provided by the processor 270.

The display 264 can be any suitable display device that outputs visual information. For instance, the display 106 can be an LCD or LED, or a touch screen. The display 264 can provide notifications and display measurements, operating parameters and/or guidance images depending on the components/functionality of the control unit 250. For example, the processor 270 may be configured to display operating parameters such as the measured resistance, the injection pressure and/or the aspiration pressure on the display 264.

The user interface 274 enables a user to provide inputs to the control unit 250 via one or more input devices, which may include, but is not limited to, the on/off button 252, the injection operation button 254 and the aspiration button 256. In some cases, an input device might be a slider, a button, a lever, a dial or a thumbwheel which may be used to adjust light intensity if the control unit 250 includes a light source used for visual guidance.

The device interface 276 can include a communication port including any combination of at least one serial port, at least one parallel port and at least one USB port that provides USB connectivity, a networking interface device, an analog to digital converter (ADC) or a digital to analog converter (DAC). The device interface 276 is used to communicatively connect the processor 270 to various devices which allows the processor 270 to send and receive signals with these other devices that may be internal or external to the control unit 250 such as the one or more sensor(s) 278 or the actuator control input 262. Accordingly, the device interface 276 may be adapted for receiving measurement data and transmitting control signals for operation of the device.

In at least one embodiment, the control unit 250 may include a communication unit that can include a radio for wireless communication by utilizing CDMA, GSM, GPRS or Bluetooth protocol according to standards such as IEEE 802.11a, 802.11b, 802.11g, or 802.11n or other wireless communication protocol. The communication unit can be used by the control unit 250 to communicate with other devices, computers or embodiments of the probe which have a corresponding radio. Accordingly, the communication unit can provide the control unit 250 with a way of communicating wirelessly with various devices that may be remote from the control unit 250.

For example, the one or more sensor(s) 278 can include the resistance sensor 107 so that the measured resistance values can be received via an ADC and analyzed by the processor 270. Likewise, the actuator control input 262 may generate an analog signal that is converted by an ADC in the device interface 276 into a digital signal that can be received and processed by the processor 270. As another example, the processor 270 can generate a digital pump control signal in response to receiving the actuator control signal, and the digital pump control signal may be converted to an analog signal via the DAC in the device interface 276 and sent to the pump(s) 280.

In another example, the speaker 268 can be communicatively coupled to the processor via a DAC in the device interface 276 and the processor 270 is configured to generate audio signals and output the audio signals via the speaker 268 where the audio signals include speech, tones or beeps corresponding to the operating parameters and/or the measurement data including the measured resistance, the injection pressure and/or the aspiration pressure. Alternatively, or in addition thereto, in at least one embodiment, a vibrator may be included in the control unit 250 and used to communicate certain operational situations such as, for example, the location of the needle tip relative to a desired position in the eye, to the user as described previously.

In another example, in embodiments where the control unit 250 includes the light source (various example implementations of which were described previously), and the device interface 276 can include a light port that is coupled via an internal optical conduit (e.g., optical fiber) to the light source 282. This then allows the light source 282 to be coupled to an external optical fiber that is connected to the light port and the probe 101 or 201.

In another example, in embodiments where the control unit 250 includes an OCT device 284 or other light imaging device as explained previously, the device interface 276 can include a light port for receiving an optical fiber that is optically coupled to an optical conduit at the probe 101 or 201, and the control unit 250 can include an internal light conduit that is optically coupled to the light port and the OCT device 284 or other light imaging device. In such embodiments, OCT measurements that are generated by the OCT device 284 or images produced by other light imaging devices may be output on the display 264 under the control of the processor 270.

In another example, in embodiments where the control unit 250 includes an US imaging device 286 and the probe 101 or 201 includes an US transducer that is disposed at a distal end of the probe 101 or 201, the device interface 276 can include an electrical port for receiving an electrical cable that is connected to the US transducer and used to send US signals generated by the US transducer. The port is connected to an internal wire that is connected to the US imaging device which receives the US signals and processes the received ultrasound signals to generate US images. In such embodiments, US images that are generated by the US device 286 may be output on the display 264 under the control of the processor 270. Alternatively, or in addition thereto, the control unit 250 may include a transducer for generating acoustic signals and/or an acoustic reflection measurement device for receiving acoustic reflection measurements, processing the received acoustic reflection measurements, and displaying the processed acoustic reflection measurements on the display 264, and the device interface 276 may include an acoustic port for coupling acoustic signals, and the probe 101 or 201 may include one or more acoustic waveguides.

The power supply unit 287 may be a power adaptor or a rechargeable battery pack depending on the implementation of the control unit 250 as is known by those skilled in the art. In some cases, the power supply unit 287 may include a surge protector that is connected to a mains power line and a power converter that is connected to the surge protector (both not shown). The surge protector protects the power supply unit 287 from any voltage or current spikes in the main power line and the power converter converts the power to a lower level that is suitable for use by the various elements of the control unit 250. In other embodiments, the power supply unit 287 may include other components for providing power or backup power as is known by those skilled in the art.

The memory unit 272 includes volatile and non-volatile storage such as ROM, one or more hard drives, one or more flash drives and/or some other suitable data storage elements. The non-volatile storage may be used to store software instructions, including computer-executable instructions, for implementing the operating system 288, the programs 290, the control application 292 and other software modules, as well as storing any data used by these software modules. The operating system 288 and the programs 290 may include software instructions for performing basic operations and functions of the processor 270 and the control unit 250. The data may be stored in the files 296, such as for data relating to operating parameters of the control unit 250 and any images when the OCT device 284 or US imaging device 286 are used. The control application 292 includes software instructions for providing the various functions of the control unit 250 described herein. The processor 270 is configured to perform these functions when the processor 270 executes the software instructions of the control application 292. The I/O (input/output) module 294 includes software instructions that, when executed by the processor 270, can configure the processor 270 to store data in the files 296 and/or retrieve data from the files 296.

In at least one embodiment, in one application, distal end of the probe 101 or 201 may be positioned posteriorly in the region of the macula and used to place a choroidal (and/or macular) buckle for treating a myopic macular hole and/or myopic tractional maculopathy. However, this may require a conjunctival cutdown to access this region. For example, a conjunctival cutdown may be carried out in the inferior temporal quadrant of the eye under local anesthesia. The intermuscular septum may also be minimally dissected. One of the probes described herein may then be passed posteriorly to the macular region. Once the probe is visualized internally to be depressing the macular region, the treatment fluid may be injected into the suprachoroidal space to create a temporary choroidal buckle in this region. In at least one embodiment, one of the probes described herein may be used to deliver certain treatments to the subretinal space in the macular region such as gene therapy or other drug solutions or drugs on extended release platforms/hydrogels.

In at least one embodiment, any of the devices described herein may be used in the clinic or in the Operating Room (OR) alone or: a) in combination with vitrectomy, b) with a chandelier for visualization and then placement of a choroidal buckle, c) in combination with pneumatic retinopexy (in the office or in the operating room) to supplement the action of the gas bubble or to address tears or holes that are inferior that cannot be adequately addressed by the gas bubble due to limitations of patient positioning, and/or d) in combination with traditional scleral buckling, where the ST procedure may be added as an adjuvant to a scleral buckle that has already been placed, either at the same time or later as a rescue procedure if the primary scleral buckle is not working. The procedure may also be used as an adjuvant to vitrectomy when there have been iatrogenic retinal tears.

In at least one embodiment, the injection conduit 104 of the probes 101 or 201 may be filed with liquid nitrogen for performing cryopexy to repair a retinal tear/detachment. Alternatively, an additional injection conduit may be added to the probes 101 or 201 and used for cryopexy while the other injection conduit may be used for injection of treatment fluid. Currently, without cryopexy, once the retina is reattached following the injection of treatment fluid into the SCS, the patient requires laser retinopexy in the ensuing days to cause a permanent chorioretinal adhesion to form between the retina and the retinal pigment epithelium around the retinal break. Alternatively, in a probe that includes cryopexy, the probe may be used to perform cryopexy first, then subretinal fluid drainage if required followed by injection of treatment fluid into the SCS. This may have the advantage of the patient potentially not requiring anything further to treat their retinal detachment which advantageously only requires one session for the procedure with no follow up sessions for further treatment.

In at least one embodiment, the injection conduit 104 of the probes 101 or 201 may include inert gas or air which may be injected intravitreally by extending the needle into the vitreous. Alternatively, an additional injection conduit may be added to the probes 101 or 201 and used for intravitreal injections. Accordingly, the same probe may be used for the ST method combined with pneumatic retinopexy. For example, the intravitreal injection may be made approximately 4 mm posterior to the limbus, where the pars plana is and where it is safe to perform intravitreal injections.

Currently, conventional devices for suprachoroidal (or subretinal) drug delivery largely depend on a scleral cutdown and then passing a probe/cannula through the suprachoroidal space to the desired target location before initiating the injection into the suprachoroidal (or subretinal) space. Some of these devices rely on the injection of the drug into the anterior suprachoroidal space from which the drug is expected to dissipate/diffuse to the target site. However, none of the conventional devices allow for direct scleral penetration at a posterior target location or have a needle which can be retracted while the device is being positioned and then variably (and controllably) extended to the desired depth. One issue preventing direct scleral penetration is that in a typical design for conventional devices the needle extends from the distal end tip of the distal end of the device, typically along (i.e. parallel to) the longitudinal axis of the distal probe end, which makes it difficult to orient the conventional probes at the correct location and with the correct angle to penetrate the sclera in the appropriate manner (e.g., approximately perpendicular or at any desired angle).

However, this challenge is overcome by the various probe embodiments described in accordance with the teachings herein where, in a first aspect, a side surface of the distal end of the probe is preferably curved with a radius of curvature similar to that of the eye (e.g., sclera) where the needle will be inserted. Since the target location for insertion may be at different parts of the globe of the eye, there may be different arc angles and radius of curvature of the distal curved portions that may be used so that the needle, exiting from the exit location of the curved side surface of the distal end of the probe that is placed adjacent to the ocular outer surface, is inserted into the sclera at approximately 90 degrees, which allows for precise localization of a target insertion location. For example, more anterior target locations at the vitreous base or the equator for applications such as retinal detachment repair or drug delivery will require a smaller distal probe end length and corresponding arc angle such as about 10 degrees to about 15 degrees (or more or less in which case a probe with a longer or shorter distal end may be used). However, for more posterior drug delivery such as close to the macula, a larger arc angle such as about 60 degrees to about 75 degrees may be preferable for the distal curved portion of the probe to allow the device to be appropriately placed on the scleral surface.

In a second aspect, the distal end portion of the probes described in accordance with the teachings herein may have an approximate flat profile for the surface of the distal probe end where the needle is extended from such that this probe surface is tangential to the scleral surface at the point of needle entry.

In a third aspect, the needle at the distal tip of the probes described in accordance with the teachings herein is extendable from a location on a side surface of the distal end portion of the device, approximately perpendicular to the longitudinal axis of the distal end portion, such that the needle may be inserted approximately perpendicularly to the scleral surface. For example, the needle may be extendable at a location on the curved surface of the distal end portion of the probe or at the tip of the probe. In either case, the needle is extendable from a curved surface of the distal end portion of the probe facing the eye such that the curved surface of the distal end portion of the probe can be placed along a curvature of the eye and the needle can enter the sclera perpendicular to the tangent of the globe at that location. Advantageously, this reduces/minimizes the distance the needle has to travel to reach the desired target location in the eye which reduces the risk of complications such as hemorrhaging. Also, the predictable straight path that the extended needle follows that is perpendicular to the tangent of the globe allows for a more predictable needle depth penetration which advantageously increases the chance of the needle tip being at the correct location and depth (e.g., the subretinal or suprachoroidal space depending on the ophthalmic procedure being performed).

In some embodiments, the probe and needle may be configured such that the needle enters the sclera at an angle that is not approximately perpendicular to the sclera at the point of entry. Such a configuration may be preferable in some cases to allow the needle tip to enter the SCS at an angle so that it will have a longer distance to travel to traverse the SCS and therefore be less likely to traverse the SCS and penetrate the choroid inadvertently.

In at least one embodiment, the three above-noted aspects with one or more of the sensing mechanisms described previously will allow for an accurate and precise localization of the needle tip at the desired target location from an external scleral location. Furthermore, the sensing mechanisms such as, but not limited to, a light beam, and/or resistance sensor (which may be implemented using a pressure sensor, a flow sensor and/or an impedance sensor), for example, are also directed in the same direction as the needle from the side surface of the distal end portion of the probe relative to the scleral surface.

Referring now to FIG. 3A, shown therein are several embodiments of a distal end portion of probe 300a, 300b, 300c with varying arc angles and arc lengths of the curved distal end portion for localization at different locations on the eye in accordance with the teachings herein. Each of the probes 300a-300c have distal end portions 302a-302c that are curved such that the lower surfaces 304a-304c of the probes 300a-300c (i.e. the surface of the probe facing the sclera during use) have a radius of curvature that corresponds to the region of the eye where the entry point must be precisely determined such that the curved distal end potions are placed conformably against the scleral surface and there are different angles between a first longitudinal axis of the probe body and an approximate second longitudinal axis of the curved distal end portion and optionally different lengths of the curved distal end portions for reaching different scleral locations. For example, the radius of curvature for the distal end portions of different probe embodiments may be approximately the same to the radius of curvature of the human sclera. For example, each probe has a radius of curvature of about 10 mm to about 15 mm in the distal curved portions. Using probe 300a, this curved section is the lower surface of the distal curved portion where reference number 304a is pointing. The arc length is the length of the arced portion of the distal end portion and is shown as 304L. The arc angle is the angle indicated as beta. The dashed pie shape is just for illustration purposes and indicating the arc angle and arc length properties of the distal end portion of the probe. The arc angle of the distal curved end portion may be about 60 degrees for probe 300a, about 30 degrees for probe 300b and about 15 degrees for probe 300c. Using probe 300a, the longitudinal axis A1 of the probe body and the longitudinal axis A2 of the distal curved end portion have an angle alpha (located as shown in FIG. 3A), which shows how the end portion is curved away from the longitudinal axis of the main body of the probe.

It should be noted that the surface of the distal end portion of the probe that is facing the eye in normal operation, and from which the needle protrudes, may be referred to as the inner surface, the lower surface, the concave surface, the side surface, or any similar term herein.

As noted, the probes 300a-300c and the curved distal end portions 302a-302c have different arc lengths for allowing the needle to be located at different target regions on the globe of the eye. Accordingly, the distal end portion has a predetermined radius of curvature and length for accessing a desired location on the eye surface during use. For example, a distal tip end portion having an arc angle of about 10-20 degrees may be used for anterior ocular locations, while an arc angle of 20-40 degrees may be used for mid-peripheral locations and an arc angle of about 50-70 degrees may be used for more posterior regions such as in the macula.

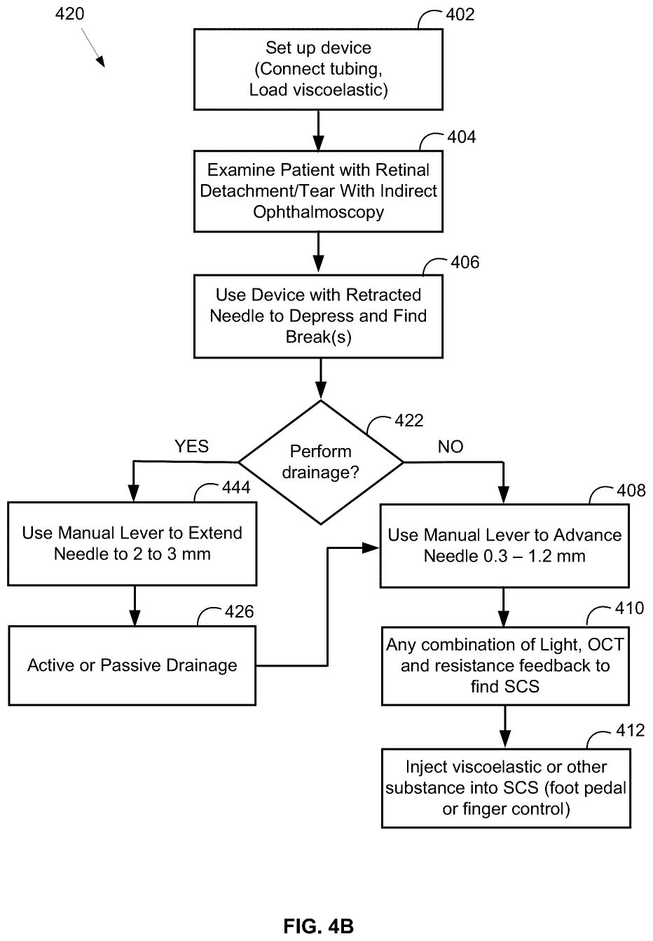

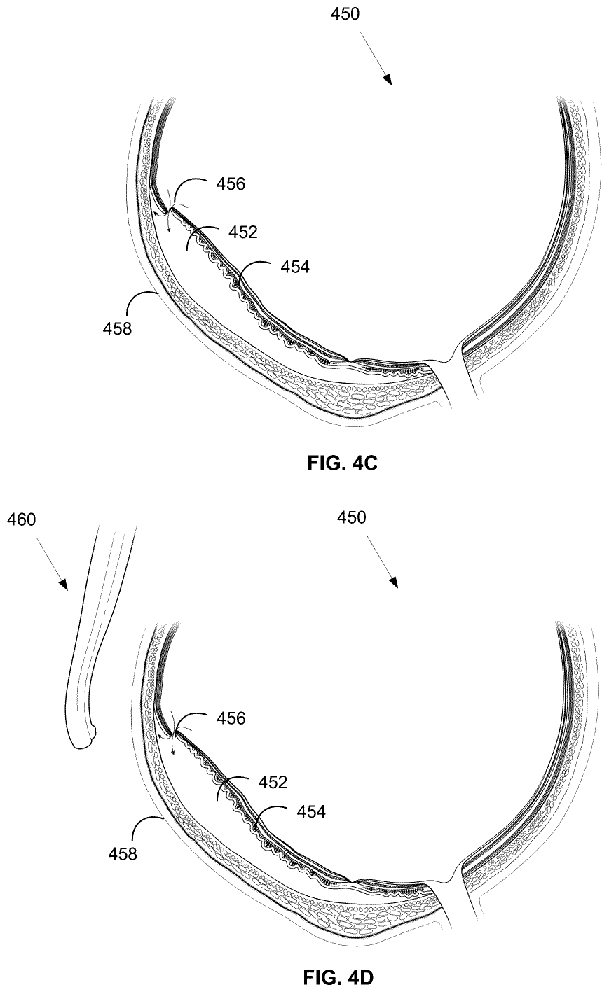

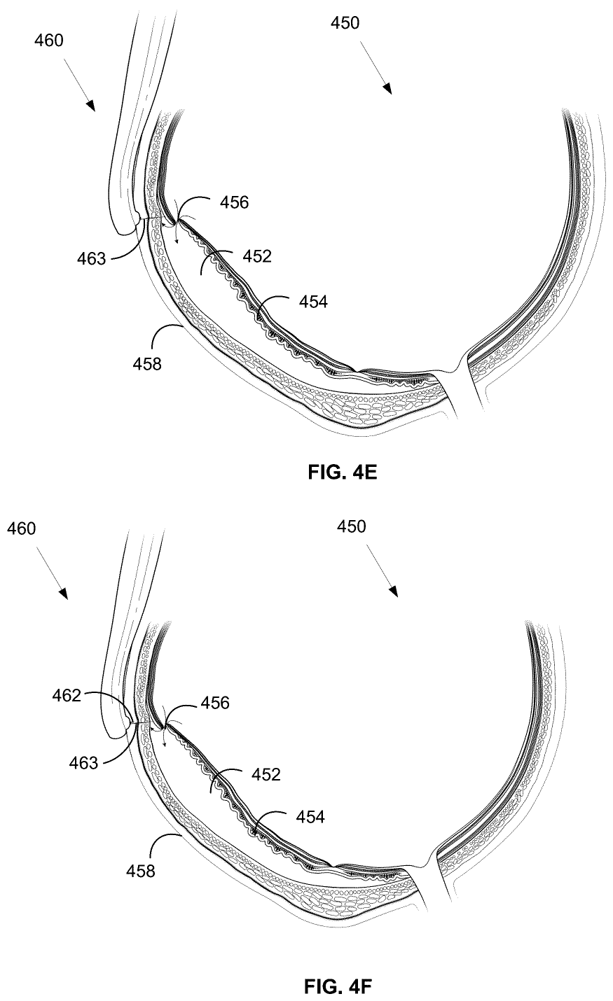

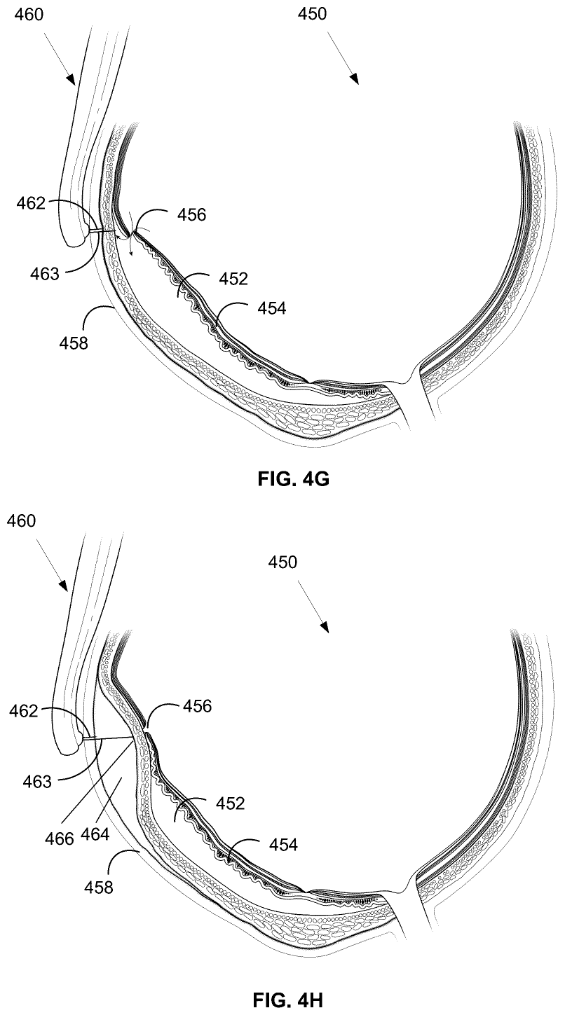

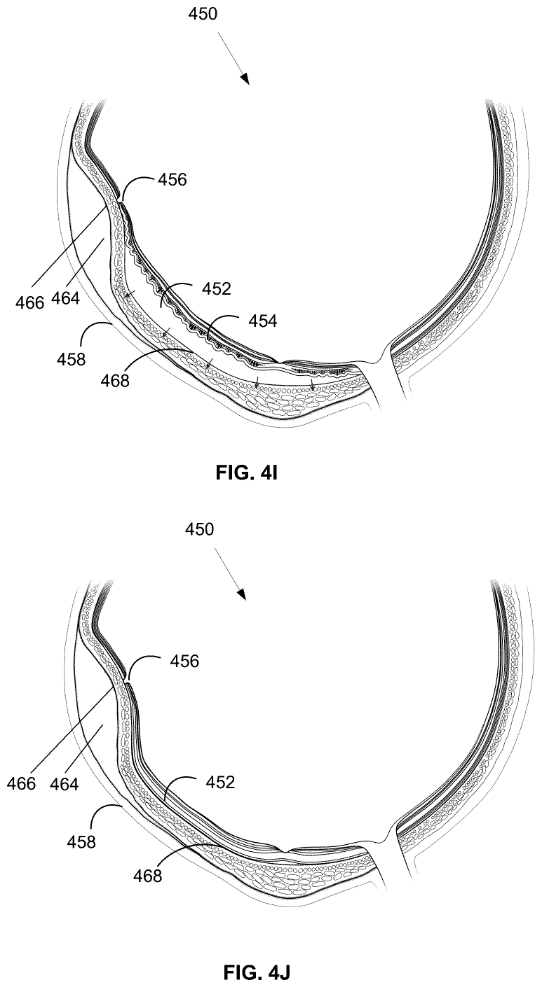

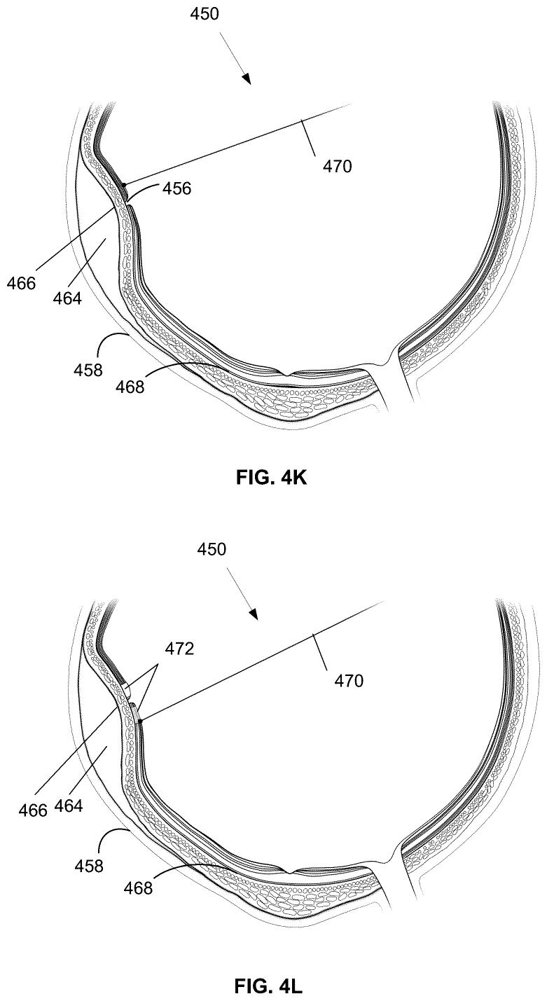

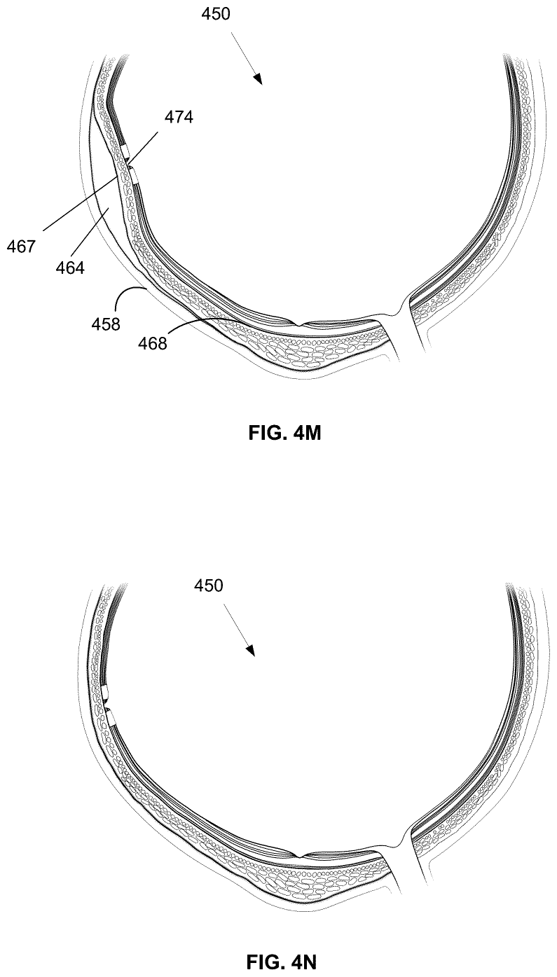

Referring now to FIG. 3B, which is a magnified view of probe 300a, it should also be noted that each probe 300a-300c may include a boss 308a-308c on the lower surface of the distal end portion from which the needle is extended. The one or more sensing elements that may be used may be directed towards the scleral surface from the boss, for various purposes such as for precise depth determination during use. For example, the guiding light beam may emanate from the boss (e.g., see 463 in FIG. 4E). Alternatively, there may be embodiments where the boss is not used but the needle and one or more sensing elements are located in a similar fashion on the lower surface of the probe. The boss 308a-308c, which may also be referred to as a protrusion, mound or nub, may be used to indent the sclera during localization of the target injection area so that the probe 300a-300c can be accurately located on a region of the scleral surface of the eye where the injection is to be made. However, the indentation of the eye surface still can be performed when the boss is not used. The needle may be extended out of the boss or a location adjacent the boss, depending on the embodiment, during use. Accordingly, the side surface of the probe has a boss at the exit location and the needle is configured to extend and retract through the boss, or the side surface has a boss adjacent the exit location and the needle is configured to extend and retract adjacent to the boss. It should be noted that any probe described herein may have the shape of the probes 300a-300c shown in FIGS. 3A-3B in various embodiments. Accordingly, the boss may be used as a visual indicator of where the needle will extend from. The dimensions of the nub may be selected such that it encompasses the dimensions of the needle that is used. For example, a boss may have a diameter in the range of about 0.5-1.5 mm when a 30 G needle is used.

In at least one embodiment, a force sensor may be included in the boss so that the user is provided with force feedback of how much force the user is applying to the surface of the sclera when extending the needle into the eye. This force feedback may be used by the user to avoid applying too much force during needle extension which may otherwise indent the sclera and make the penetration depth be deeper than desired.

In at least one embodiment, the boss may be used to provide more room to house the needle and one or more sensors without changing the thickness of the entire distal probe end.

In at least one embodiment, the boss may be moveable or vibrate during use and this may be used to help identify the location of the distal end portion of the probe and where the needle will extend from the probe during use. For example, the boss may be moved in a reciprocating/oscillating fashion at a certain frequency (e.g., rhythmically), and this motion may be visible on the inside of the eye while thereby assisting with visualization and identification of the position of the curved distal end portion of the probe.

In at least one embodiment, the amount by which the boss protrudes on the distal portion of the probe, or the boss location may be adjustable by mechanical, electrical, or pneumatic means. In a first position, the boss may be flush with the lower surface of the distal end portion of the probe or recessed from this surface such that the boss does not contact and does not apply pressure to the eye even when the probe is in contact with the eye, as may be preferable while the probe is being advanced along the outside of the eye toward the desired position. In order for the user to better visualize the position of the probe, the boss may be adjusted at times to protrude from the lower surface of the probe distal end portion and apply pressure to the eye, causing an additional indentation (in addition to the indentation caused by the lower surface of the distal portion of probe which is in contact with the eye/sclera) which may be visible to the operator from outside the eye by observation through an ophthalmoscope or other means. In at least one embodiment, the boss may be adjusted to protrude more or less, and cause a larger or smaller indentation, as may be needed to facilitate observation.

In any embodiments of the probe described herein, the distal curved end portion may be rigid. Alternatively, in at least one embodiment, the probe may have a flexible portion proximal to the distal end portion that is adjustable to adjust an angle of the longitudinal axis of the distal end portion relative to a longitudinal axis of the main body. In such embodiments the angle may be adjustable between about 15 degrees to about 60 degrees.

In another alternative, any of the embodiments described herein may be modified such that the distal curved end portion is telescopic with respect to the main body of the probe. This may aid in reaching the proper posterior location on the eye when performing the ocular procedure. For instance, using probe 300b as an example, the length L1 of the distal curved end portion may be increased by using a telescopic arm that extends approximately from location T in FIG. 3A. In at least one case, there may be a locking mechanism that is engaged so that the telescoping is not done by accident during use.

Also, any of the embodiments described herein may be modified so that the distal curved end portion may be flexible enough so that the radius of curvature of the distal curved end portion may be changed to better conform to the surface of the eye (e.g., sclera) during the ocular procedure. For instance, using probe 300b as an example, the radius of curvature for the lower surface of the distal probe end where reference numeral 304b is pointing may be slightly changed by bending the distal end portion of the probe 300b.

Referring now to FIGS. 3C-3D (dimensions are not to scale), shown there are examples of placement of the distal end of different probes 310 and 320 at different locations on the scleral surface of the eye. In FIG. 3B, the probe 310 has a boss 312 that, along with one or more of the sensing mechanisms described herein, allows for accurate localization and depth penetration of a needle 314 which extends from the distal end portion of the probe 310 approximately perpendicularly into the sclera and the treatment fluid is injected forming a bleb (e.g., viscoelastic bleb in this example) in the SCS which forms a choroidal buckle. However, the boss may also be optional with no impact on procedural accuracy when using the probe. In FIG. 3C, the probe 320 also has a boss 322, which can be optional, but a probe is used that has an angle between the longitudinal axes of the probe main body and the curved distal end potion and a longer length to be able to reach further back along the posterior surface of the eye so that the treatment fluid may be injected in the SCS or sub-retinal regions depending on the ocular treatment being performed. In each of these examples, during the procedure, the depression on the scleral surface of the eye is seen through one of the visualization techniques described herein to properly position the distal curved portion and the needle of the probe to indent the scleral surface of the eye before the needle is extended and starts advancing into the eye.

As seen in FIGS. 3C and 3D, the distal end of the probe is angled and radius of curvature that conforms to the eye (e.g., scleral surface). The probe can be placed directly on the conjunctiva as far back as possible based on the conjunctival fornix. For more posterior locations a small conjunctival cutdown may be required. Once the probe is placed on the scleral surface at the desired location, the surgeon can gently indent with the device and this indentation created by the distal portion (boss or plate portion depending on embodiment) of the probe is visible internally. The needle exit site (e.g., exit location) can be identified by assessing the midpoint of the indent (since the needle exit site will be in the middle of the distal portion of the probe). Therefore, the surgeon can place the internal target location on the midpoint of the indent to center the choroidal bleb appropriately. This procedure can be done with topical, subconjunctival, subtenon's or retrobulbar anesthesia. In most cases topical or subconjunctival anesthesia will be sufficient. The appropriately curved distal part of the probe, together with the needle coming out perpendicular from the inner surface of the distal portion of the probe allows the surgeon to navigate to various target regions on the conjunctival or scleral surface, and not be impeded by the orbital and periorbital tissue, so that the needle can penetrate the conjunctiva and/or sclera at the correct angle. While the description may have described the probe contacting the scleral surface, without a conjunctival cutdown, the lower surface of the probe may be contacting the conjunctival surface (which is the thin mucous membrane covering the episclera and sclera). Also, as explained earlier, a guidance light beam 324 may be used to assist with proper positioning and insertion depth of the extended needle. For example, the guidance light beam may be used for illumination or to indicate when a tip of the needle 106 penetrates into different layers of the eye based on determining changes in transmitted or reflected light.

In at least one embodiment, a first safety mechanism is included in any of the probes described herein by including a sensor that is used to sense how much force the user is placing on the distal end of the probe that is in contact with the eye. Feedback may then be provided to the user of the amount of this force so that the user knows not to apply too much force as this may result in the needle penetrating too deeply into the eye and injecting fluid into or removing fluid from an incorrect location in the eye. Exerting too much force may also lead to other undesirable outcomes such as hemorrhage or retinal tears, for example. Any suitable force sensor that is sensitive enough to measure the force and/or pressure at the portion of the probe that contacts the eye may be used. When the amount of measured force is larger than a force threshold, which indicates that the needle may start to penetrate too deeply into the eye, feedback may be given to the user, such as a sound feedback, speech feedback or another suitable form of feedback, for example, to notify the user not to push any harder.

In at least one embodiment, a second safety mechanism is included in any of the probes described herein to protect against the case where too much force is being exerted upon the patient's eye from the distal end portion of the probe. Referring now to FIG. 3E shown therein is an example embodiment of a portion of a probe 330 with a pressure distribution flange 332. The pressure distribution flange 332, which may be implemented by using dish-shaped medical grade half-sphere or membrane, acts as a guard and is positioned on the lower surface of the probe 330 that makes contact with the scleral surface of the eye. The shape of the surface of the flange 332 that contacts the scleral surface of the eye may be concave and have a radius of curvature such that it conforms to the shape of the eye (e.g., scleral surface) where the needle is inserted. The flange 332 may come in different sizes and/or different degrees of concavity to be able to suit different sized eyes. The flange 332 may be a separate piece that is attached to the probe 330 (such as by a snap-fit connection) or the flange 332 may be integral with and part of the probe 330. Preferably, the flange 332 is located so that a needle 334 extends from an approximate center location on the flange 332. For embodiments that include the guidance light beam, the flange 332 may be made of a material that is transparent to the light wavelengths that may be used for the guidance light beam so that the guidance light beam is shone through the flange 332 without much attenuation or the flange may have an aperture through which the guidance light beam is shone during use. The flange 332 may be located to be adjacent and above a boss 336 so that the flange 332 is between the boss 336 and the scleral surface of the eye during use. However, in embodiments that do not use the boss, the flange 332 is positioned directly on the lower surface of the distal end portion of the probe.

In at least one embodiment, the flange 332 (which may also be referred to as a guard) may change in shape when the user is applying too much force, which if the flange 332 were not used, would cause the needle tip to extend to a larger depth than desired so that the treatment fluid is not injected at the correct location. Accordingly, when the localization is correct and the sclera is being indented, this feature of the flange 332 may aid in maintaining the amount of force at a relatively constant level while the needle 334 is then extended, otherwise the needle may advance too deep. In at least one embodiment, a change in the shape of the flange 332 can be used as a visual cue to notify the user to apply a smaller amount of force. Alternatively, or in addition thereto, the flange 332 may be formed so that the force exerted by the user will be distributed over the surface of the flange 332 to protect against the user applying too much force that would be otherwise concentrated at the boss 336 (if used) or along the surface of the probe around the base of the needle 334 such that the sclera may be indented and the needle 334 may penetrate deeper than intended when the needle 334 is being extended. Also, the flange 332 may be dimensioned to allow for a small indentation but prevent a larger indentation from too much force that may cause too much penetration depth into the eye. The flange is also preferably sized so that it is not too large to obstruct the user's field of view. In at least one embodiment, the same flange can be used to indent the globe and replace the function of the boss 336 such that the boss 336 is not needed.

In at least one embodiment, a third safety mechanism is included in any of the probes described herein to protect against the case where too much force is exerted such that the needle tip is extended too deep into the patient's eye. The third safety mechanism may be used alone or in combination with the first safety mechanism and/or the second safety mechanism. The third safety mechanism includes a depth limiter, such as a post or annular disk that is within the distal portion of the probe so that the needle cannot extend past the surface of the distal end portion of the probe that contacts the scleral surface by a depth limit amount which may range from about 0.5 mm to about 1.5 mm and be changed based on the particular eye (e.g., due to differences in scleral thickness) and from about 1 mm to about 3 mm depending on the location of injection and the function (e.g., first injection, second invention, or drainage), for example.

In at least one embodiment, a fourth safety mechanism is included in any of the probes described herein to protect against the case where too much force is being exerted upon the patient's eye from the distal end of the probe. Referring now to FIG. 3F, shown therein is an example embodiment of a portion of a probe 340 which includes a variable coupler 342. The variable coupler 342 is disposed between the boss 336 and the flange 332. In some embodiments having the variable coupler 342 and the flange 332, the boss 336 may be optional. Also, in some embodiments with the variable coupler, the flange 332 may be optional and the variable coupler may be located underneath the boss 336. The variable coupler provides a variable mechanical coupling to avoid situations in which the user is applying too much force such that the needle penetrates too deeply. Accordingly, in at least one embodiment, the device/probe includes a flange and/or a variable coupler at the exit location to maintain a position or a pressure between the side surface and the surface of the eye. In at least one embodiment, the variable coupler may include a spring or have a piston that moves within a cylinder to absorb some extra force. In at least one embodiment, the spring may be adjustable such that the coupling is configured to be rigid during needle insertion but then become ‘soft’ once the needle tip is at the desired depth so that further changes in applied pressure do not result in changes in the needle tip position. In at least one embodiment, the variable coupler 342 may be adapted to dampen pressure applied by the distal end portion of the probe to the eye when a pressure or force applied to the eye during use is higher than a threshold. For example, the variable coupler may include a pressure release valve which releases pressure when the applied pressure to the eye during use is higher than the threshold or the variable coupler comprises a force damper when force applied to the eye during use is higher than the threshold.

Referring now to FIG. 3G, shown therein is a front view of an example embodiment of a pressure distribution flange 350 with one or more sensors 352 (only one of which is labelled for ease of illustration and shown as dots for simplicity). In at least one embodiment, the sensor(s) 352 may be a strain sensor that is used to detect a change in curvature of the flange 350 which would indicate that too much force is being exerted by the user during scleral indentation or needle extension, and feedback (such as audio or visual) is provided to the user so that they can reduce the exerted force. Alternatively, at least one of the sensor(s) 352 is a pressure or force sensor whose measurements can be compared to a force threshold to provide audio or visual feedback to the user when too much force is being exerted during scleral indentation and/or needle extension. In another alternative, there are multiple pressure/force sensors distributed uniformly around the flange 350, if the sensors 352 provide approximately similar measurements this can indicate that the flange is normal to the globe during scleral indentation such that the needle 334 is being extended approximately perpendicularly into the sclera, and if the measured pressure is uneven it may indicate that the flange is tilted such that the needle 334 may not be inserted perpendicularly into the sclera if the needle 334 were extended/deployed. This may be useful when performing manual insertion of the needle into the eye.

Referring now to FIG. 3H, shown therein is an example embodiment of an ocular device 360 that can be used with an ocular treatment device for accurate localization and precise needle depth penetration for ocular treatments. In this example embodiment, the ocular device 360 is fully contained within the main body 102 so that the ocular device 360 also functions as the probe such that it is a standalone device and does not use any external fluidic connections (e.g., tubing) or electrical connections. In at least one embodiment, the hardware of the device 360 may be contained in a first portion and the fluidics may be contained in a second portion that is releasably connectable to the first portion. This allows for the second portion housing the fluidic components to be disposable for single use in a single patient or partly disposable with some reusable components that can be sterilized. The distal end portion generally has a curved portion with an arc angle as explained for other embodiments of the probe described herein.

The standalone ocular device 360 may have various internal structures and in this example, the ocular device 360 has some similar components as the probe 201 and also includes several components of the control unit 250. All of these similar components operate as previously described. It should be noted that the elements are not drawn to scale in FIG. 3H. In at least one alternative embodiment, a boss may be included at the distal curved end portion as explained for other embodiments described herein.

The ocular device 360 may perform both injection and drainage and includes a drainage conduit/tube 228′ that is disposed to one side of the device 360 while hardware is disposed on the other side of the device 360. In an alternative embodiment, the device 360 may include just a single conduit, such as conduit 104, that may be used for both injection and drainage as described for another embodiment herein. A port 210 is also available for connection to a plunger if needed to inject fluid into the eye (or remove fluid from the eye when the conduit 104 is used for both injection and drainage). Otherwise, in dual conduit implementations, the drainage conduit 228′ is coupled to a drain port 362 which may be fluidically coupled to a drainage device (e.g., a pump). The drain port 362 may be optional in cases where the fluidic portion of the device 360 is discarded after use. The device 360 also includes a fluid port 216 for coupling fluid into the injection conduit 104 where the fluid is to be injected into the eye during use. Alternatively, the device 360 may come preloaded with the fluid, or may receive a fluid cartridge, as described for other embodiments herein.

In this example embodiment, the device 360 includes a microcontroller 362 for controlling the operation of various hardware elements of the device 360 and a power source 364 for providing power to various components of the device 360 such as the microcontroller 364 and any motors, for example. The microcontroller 364 also includes a memory device (not shown) for storing program instructions that configure the microcontroller 364 for performing various functions when the program instructions are executed by the microcontroller 364. The power source 366 may be a battery which may or may not be rechargeable. If the device 360 is instead tethered to a power outlet or another device that can provide power, the power source 366 may include surge protection and voltage regulation circuitry.

The device 360 also includes a mode selection input such as a button, slider or the like to toggle between injection mode in which fluid is injected into the eye and drainage mode in which fluid is drained from the eye. The device 360 also includes a fluid actuation control input 370 such as a button slider or the like to control an internal fluid actuator (not shown) to perform the injection or drainage where the internal fluid actuator may be a motor that pushes/pulls on an internal plunger (not shown) in the various conduits. The fluid actuation input 370 may be pressed while the fluid is injecting or draining and then released to stop the injection or drainage depending on whether injection mode or drainage mode is selected.

The device 360 also includes a needle actuation control input 372 which may be a slider, rotary dial or other input mechanism that is used to control the extension and retraction of the needle 106 as explained previously for other embodiments described herein. The input 372 may be used to gradually extend the needle into the eye as described herein.

The device 360 may also include one or more sensors 107 such as any combination of a pressure sensor, a force sensor and an impedance sensor as explained earlier. The location of the sensor 107 is shown for illustrative purposes in FIG. 3H and may be at other locations in other embodiments.

The device 360 also includes a light source 374 which may be used for generating white light that is transmitted along the optical fiber 109 and shone out of the lower surface of the distal end portion adjacent the location where the needle 106 is extended away from the lower surface of the distal end portion during use. The light source 374 may generate a white light during visualization or a colored light beam that is used as a guidance light beam as explained in other embodiments described herein. The light source 374 may be at the distal end of the device 360 and be coupled to the injection conduit more distal and use the injection conduit as a light transmission conduit. In at least one embodiment, mirrors may be used to reflect the laser/light along the length of the device 360.

In at least one embodiment, the device 360 may also include a radio (not shown) for short-range or long range wireless communication so that any sensor data that is collected during operation can be provided to another device, such as a display or the control unit 250 for providing feedback of various parameter values and feedback to the user during operation such as was described for control unit 250.

In at least one embodiment, the device 360 may include one or more output devices such as a small display (not shown) such as an LED display, for example, to provide visual feedback to the user of values for certain parameters during operation of the device. In at least one embodiment, the device 360 may include a speaker and/or vibrator for providing feedback to the user as was explained in earlier embodiments described herein. In at least one embodiment, the vibrator and/or speaker may be optional.

In an alternative embodiment, the device 360 may be constructed to not use any electronic hardware in which case the implementation of the device uses mechanical and/or pneumatic elements for control and actuation.

Referring now to FIG. 4A, shown therein is a flowchart of an example embodiment of a method 400 for treating retinal tear or RRD in accordance with the teachings herein. At step 402 of the method 400, the user sets up the ocular treatment device with the probe 101 or 201 or another suitable probe described herein. The setup may include connecting the various tubing and electrical wires and loading the treatment fluid.

The method 400 then proceeds to step 404 where the user examines the patient having the eye with a RRD or retinal tear with indirect ophthalmoscopy or wide-field viewing in the operating room. For example, the posterior segment of the eye may be visualized using binocular indirect ophthalmoscopy. An optical lens may be used at this time such as a 28D lens or another lens such as a 20D lens. A light source may be worn by the user on their head so that they are able to view the inside of the eye binocularly so the user has a 3D view. The user may use this equipment when examining the back of the patient's eye and during treatment of the retinal tear or RRD.

The method 400 then proceeds to step 406 where the user uses the probe 101 or 201 with the needle retracted so that it does not extend past the distal end of the probe 101 or 201 so that the scleral end of the probe 101 or 201 or another suitable probe described herein may be used to depress the sclera and find the break(s) in the retina. One of the localization/visualization techniques described herein may be used in method 400 to ensure that the needle is at the proper location on the globe and being inserted approximately perpendicular into the sclera.

Once the user finds the break(s) in the retina, the method 400 proceeds to step 408 where the user may use the needle actuator 108 to extend the needle 106 to a depth of about 0.3 mm to about 1.5 mm to reach the break in the SCS. One of the techniques for extending the needle described herein may be used, which may include using any combination of one or more sensors, one or more visualization techniques, one or more safety mechanisms described herein including the motorized or graduated manual slow advancement of the needle with an injection pressure applied.

The method 400 then proceeds to step 410, which is optional. At step 410, depending on the implementation of the guidance tool for the probe 101 or 201 or other suitable probe described herein, any combination of light, OCT measurements, US imaging and resistance feedback may be used to confirm that the tip of the needle 106 is located in the SCS.

The method 400 then proceeds to step 412, where the user injects the treatment fluid, such as a viscoelastic agent or other substance, into the SCS of the patient as described earlier. The user keeps injecting the treatment fluid until a sufficient amount is injected. For example, a sufficient amount may be injected once a choroidal bleb/buckle is formed all around the retinal break(s) and extends beyond the edges of the retinal break such as, for example, but not limited to, about 4 mm to about 5 mm beyond the edge of the retinal break(s) in all directions. The volume of the treatment fluid that is used may vary based on the number, size and location of the retinal breaks. For example, from about 0.7 cc to about 1 cc of treatment fluid may be injected in most cases. The formation of the choroidal buckle from a choroidal indentation under the retinal break leads to a change in intraocular fluid dynamics (i.e., intraocular fluid currents) and reduced traction on the retinal break, so that less fluid or no fluid enters the subretinal space through the retinal break and the retina then reattaches as the retinal pigment epithelium reabsorbs any fluid in the subretinal space. This choroidal buckle may be implemented using the techniques and devices described herein in any quadrant including inferior quadrants.

In an alternative embodiment, a second injection may be performed after the first injection. The second injection may be much easier to perform than the first injection because the first injection already created a separation between the choroid and the sclera. So, for the second injection, the needle can be extended to a greater length, such as about 1 mm to about 2 mm, for example. The second injection will be easier because the bleb that is already present from the first injection will make it less likely to penetrate the needle 106 into the subretinal or intravitreal space due to the larger gap in the SCS and therefore the needle can now be longer which makes it easier to manipulate and place into the correct space where the existing viscoelastic is present. In other words, the second injection of the treatment fluid may be in the location of the first bleb of the treatment fluid. This is another advantage of using a device having an extendable needle according to the teachings herein for the second injection, since the amount of extension of the needle can be tailored depending on where the second injection is needed. In some situations, it may be desirable to remove the injected viscoelastic, such as when too much viscoelastic has been injected such that it might raise the intraocular pressure too high, or if certain further treatment is required, such as vitrectomy, after performing the ST method. In these cases, the variable extension of the needle can again be used and the needle advanced to a certain extension beyond the lower surface of the distal end portion (thereby in effect varying the length of the needle used to penetrate the eye, with or without additional guidance, to enter the suprachoroidal space and then aspirate the viscoelastic. In some cases, it may be desirable to treat multiple locations in the same eye because the patient has multiple retinal tears or breaks. This can be achieved by creating smaller localized blebs around each tear or break, or by doing sequential treatments one day apart so as to not inject too much treatment fluid at one time and raise the intraocular pressure too high.

Referring now to FIG. 4B, shown therein is a flowchart of another example embodiment of a method 420 for treating a retinal tear or a RRD in accordance with the teachings herein. The method 420 is somewhat similar to the method 400 as it includes steps 402 to 412 for setting up the device and performing an injection. However, method 420 includes additional steps for performing drainage on the patient's eye before performing the injection. Since method 420 involves performing drainage, the probe 201 or another suitable probe described herein may be used. For example, step 422 may be performed after step 406 where the user uses the probe 201 with a retracted needle to depress the sclera and find break(s) in the retina. Step 422 involves the user checking to see if drainage must be performed. This might involve the user examining the patient's eye with indirect ophthalmoscopy and/or wide-angle viewing systems with a condensing lens that allow a wide peripheral view of the posterior segment. If the retina is very bullously detached, this may increase the likelihood that the user may consider adding drainage to the procedure. When draining is used, this may allow the retina to settle better on the choroidal buckle formed by the viscoelastic which will increase the likelihood of the choroidal buckle effectively closing the retinal break and reducing the flow of fluid through the retinal break and into the subretinal space. If the user determines that drainage is not needed, for example with more shallow retinal detachments, where the choroidal buckle is providing adequate indentation to close the retinal break without drainage, then the method 420 involves performing steps 408 to 412 as was described for method 400. This might also include doing a second injection later as described previously. Alternatively, if the user determines at step 422 that drainage is needed, then the method 420 moves to step 444 where the user uses the needle actuator 108 to extend the tip of the needle to a length/depth from about 1.5 mm to about 3 mm. The needle tip is extended 1.5 to about 3 mm so that the needle 106 is safely present in the subretinal space but far enough away from the retina so as to not incarcerate the retina or cause a retinal tear. The method 420 then proceeds to step 426 where active or passive drainage is performed as described previously. The user may decide to stop drainage by visually observing that the retinal detachment in the patient's eye is settling by seeing the fluid go away from the subretinal space causing the retina to flatten/attach. The user will stop drainage and remove or slightly retract the needle before the needle comes in contact with the retina. After the aspiration is performed, the method 420 proceeds to step 408 at which point the user may have to reposition the needle in the location of the retinal break before advancing it into the SCS. In other embodiments of this method, the drainage may be performed after (instead of before) injection of the viscoelastic, or drainage may be performed alone with no injection of viscoelastic.

Referring now to FIGS. 4C-4N, shown therein are images of different stages when repairing a retinal tear or RRD in accordance with the teachings herein. In FIG. 4C, a patient eye 450 demonstrating a rhegmaogenous retinal detachment (RRD) 452 with outer retinal corrugations 454. As can be seen, a probe with a boss is used in this example. However, as explained previously, a probe without a boss can be used. The patient has a temporal retinal break 456 and the arrows indicate that liquified vitreous has entered the subretinal space through the retinal break 456. In FIG. 4D, a probe 460 for the suprachoroidal delivery of viscoelastic, in accordance with the teachings herein, is advanced and accurately placed so that the needle of the probe 450 will be at the desired eye surface location at the location of the offending retinal break. In FIG. 4E, a guidance light beam 463 may be shone towards the eye surface to aid with localization. In FIG. 4F, the needle 462 starts to be extended when the user determines that the needle is at the proper location on the eye surface, and the needle 462 starts to penetrate the eye surface. In FIG. 4G, the needle 462 penetrates into the sclera 458 and some injection pressure is applied as the needle advances through the sclera. Because of the compact scleral fibers, any injection pressure will not lead to flow of the viscoelastic agent until the needle 462 enters the SCS. In FIG. 4H, once needle enters the suprachoroidal space, the viscoelastic 464 will flow from the probe 460, through the needle conduit and into the SCS creating a choroidal indentation/buckle 466 and the needle 462 may be retracted and the probe 460 removed. In FIG. 4I, the choroidal indentation/buckle 466 reduces/eliminates the flow of liquified vitreous from the vitreous cavity into the subretinal space. The retinal pigment epithelium (RPE) 468 regains control of the subretinal space and reabsorbs the subretinal fluid (as shown by the arrows). In FIG. 4J, the RPE 468 has reabsorbed most of the subretinal fluid and the retina is close to being fully attached. In FIG. 4K, the retina is fully attached and laser retinopexy 470 may be applied to form a permanent chorioretinal adhesion between the retina and the RPE 468 around the retinal break. In some cases, cryopexy may be applied to the retinal break prior to the suprachoroidal injection of viscoelastic thereby not requiring laser retinopexy later. In FIG. 4L, the laser retinopexy is completed around the retinal break. The laser burns turn into scars 472 that causes a chorioretinal adhesion. In FIG. 4M, a chorioretinal adhesion 474 has formed around the tear. The suprachoroidal viscoelastic 464 is starting to resorb resulting in a reduced choroidal indentation/buckle 467. In FIG. 4N, the retina is fully attached. There is a good chorioretinal adhesion and the suprachoroidal viscoelastic has completely reabsorbed.

It should be noted that while the example uses of the devices described herein relate to providing treatment fluid for treating a retinal tear or RRD, it should be understood that the various embodiments of the devices described herein may be used for accurate localization and precise needle penetration depths for a variety of other ocular procedures, which is especially advantageous when the location of treatment is provided from a posterior region of the eye. Furthermore, in these various ocular procedures, the treatment fluid may be, but is not limited to, a drug, a gene therapy, a hydrogel or a different type of viscoelastic fluid. For example, the devices described herein can be used for more accurate posterior targeted delivery of drugs, without relying on drug diffusion, such that the drug can be injected exactly into or adjacent to the diseased ocular tissue. Furthermore, hydrogels with sustained delivery of novel or orphan drugs may be precisely injected where needed. For example, a hydrogel or other extended release platform may be used with one of the device embodiments described herein to deliver any pharmacological agent to the posterior aspect of the eye. This includes drugs to treat conditions such as, but not limited to, age-related macular degeneration, diabetic macular edema, diabetic retinopathy, retinal vein occlusion, proliferative vitreoretinopathy, macular edema, inherited retinal diseases, intraocular tumors, posterior segment inflammation, vascular anomalies, juxtafoveal telangiectasia, Coats's disease, retinopathy of prematurity, familial exudative vitreoretinopathy and other conditions of the retina and choroid. One of the device embodiments described herein may also be used to administer treatments directly into the SCS or subretinal space where these treatments include, but are not limited to, antibody treatments, antibody fragments, aptamers, orphan drug solutions, gene therapy, and the like.

The various devices and methods described in accordance with the teachings herein are also advantageous compared to conventional drug/agent delivery systems for the subretinal space and the suprachoroidal space which rely on a scleral penetration/incision anteriorly from which a cannula/catheter may be advanced through the suprachoroidal space. Conventionally, the agent is then injected in the suprachoroidal space or a needle may be used to penetrate the choroid and the retinal pigment epithelium and inject the agent into the subretinal space. The problem with this approach is that it requires a more invasive operating room procedure and a scleral incision and the tunnelling of the cannula/catheter in the SCS. These maneuvers have inherent risk which can be circumvented, and the procedure simplified, by using one of the devices described herein that allows direct scleral penetration (either transconjunctival or with a minor conjunctival cutdown) at an accurate desired location for delivery of the agent into the SCS, the subretinal space or another portion of the eye.

The accurate localization and needle depth precision at a desired location on the globe of the patient's eye may be improved according to the teachings herein by using an appropriate curve/angle and length for the probe to reach any desired scleral location and extending the needle from a side surface of a distal end portion of the probe that is adjacent to the scleral surface so that the needle is inserted directly through the sclera (or transconjunctivally) at an appropriate angle (preferably approximately perpendicular, e.g., 90 degrees) to the sclera to allow for scleral penetration into the SCS, choroidal/RPE penetration into the subretinal space or other penetration at other regions of the eye. The accuracy may be further improved by using a guidance tool and/or one or more sensors as described herein to allow the needle to precisely reach the desired target layer (suprachoroidal or subretinal) without over or under penetration. The accuracy/ease of use of the devices described herein may be further improved by using one or more of the actuation mechanisms described herein to precisely locate the needle and the penetration depth and/o provide safety mechanisms as described herein to prevent over and under penetration of the needle and/or use of excessive force during needle insertion.

At least one of the embodiments of the devices and methods described herein may be used for one or more purposes such as, but not limited to, a) injecting viscoelastic agents in the SCS at a given location in the region of the offending retinal tears to treat RRD, b) draining subretinal fluid in the region of a RRD, c) delivering drugs into the SCS at posterior locations such as the macula, d) delivering drugs into the subretinal space at posterior locations such as the macula and e) draining of suprachoroidal fluid/hemorrhage or subretinal fluids/hemorrhage, for example.

Referring now to FIG. 4O, shown therein is a flowchart of an example embodiment of a method 480 for performing accurate localization and precise needle depth penetration on a surface of the eye for an ocular procedure. The method 480 may be performed by using one of the devices described in accordance with the teachings herein.

At step 482, setup is performed to prepare the device for use. This may involve performing calibration and also loading an agent into the device (if it is not prefilled with the agent). Any required tubing and wiring may be connected (if needed) and the device may be primed if needed. The tubing or wiring is not needed for the self-contained device.

At step 484, the device is moved by the user to place the distal end portion of at the device at the desired location on the sclera or conjunctiva by forming an indent as described previously. This can be done directly on the conjunctiva for locations at the equator or anterior to that. For locations posterior to the equator, a small conjunctival cut down may be required. It should be noted that small conjunctival cut downs are typically not significant while scleral cutdowns are more invasive. This desired location may be confirmed by indirect ophthalmoscopy (in other words the user may examine the eye internally to see the indentation created by the distal end potion to confirm accurate localization) or with wide-field viewing in the operating room. The desired location may be also confirmed using at least one of the sensors described herein.

At step 486, since the user has predetermined if the injection/aspiration is either a) suprachoroidal or b) subretinal, the user can confirm the penetration depth before inserting the needle into the eye, for example by using an imaging method such as OCT, ultrasound or other optical method, guidance tools, and/or sensing mechanisms described herein to measure the thickness of the eye layers and/or structures.

At step 488, the needle is extended into the eye, which may be done manually or using an automated technique described herein. The needle is extended from a side surface of the distal end portion of the device, which may be close to the tip of the distal end portion but fires (e.g., extends) from the side surface of the probe adjacent to the ocular surface, where the side surface is adjacent to the scleral surface with an appropriate angle/orientation through the sclera and towards the suprachoroidal or subretinal space.

At step 490, for SCS injection, injection pressure is applied as the needle advances through the sclera so that the injection takes place as soon as the needle enters the SCS preventing excessive needle depth penetration. For subretinal injection, the needle is advanced and once it is in the choroid and close to the RPE, the injection pressure is applied so that a bleb of subretinal fluid forms as soon as the needle penetrates the RPE. For aspiration from the SCS, the aspiration is started while the needle is in the sclera so that fluid will flow as soon as needle enters the SCS. For subretinal aspiration, the aspiration is started once the needle is visualized in the subretinal space. Needle localization (e.g., penetration depth) may be confirmed using one of the guidance tools and/or sensing mechanisms described herein such as, but not limited to, fiberoptic light/laser, laser reflectance, insertion resistance, etc. to confirm precise needle depth penetration.

At step 492, while the injection/aspiration is being performed in the SCS or subretinal space, the user (e.g., surgeon) may be examining the inside of the eye either with indirect ophthalmoscopy or with wide-field intraoperative viewing to assess the desired clinical endpoint.

At step 494, once the clinical endpoint is reached, the user can retract the needle or remove the needle from the eye. The intraocular pressure may be assessed by assessing optic nerve perfusion. If the central retinal artery is pulsatile or occluded, a slow anterior chamber paracentesis can be performed to lower the pressure. This may be done in small amounts, as overly reducing the pressure quickly could lead to hemorrhage.

At step 496, it is determined whether repeat procedures or reinjections/re-aspirations may be required as this may occur in some cases depending on the circumstances.

### Example 1: In-Office Suprachoroidal Viscopexy for Rhegmatogenous Retinal Detachment Repair

The results of a minimally invasive, in-office treatment application of an embodiment of the ST method of treating RRD is now discussed.

### Methods

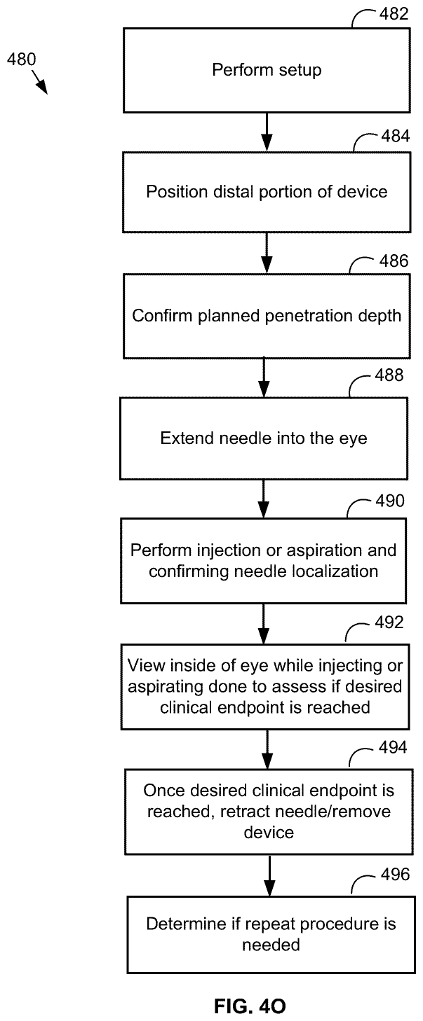

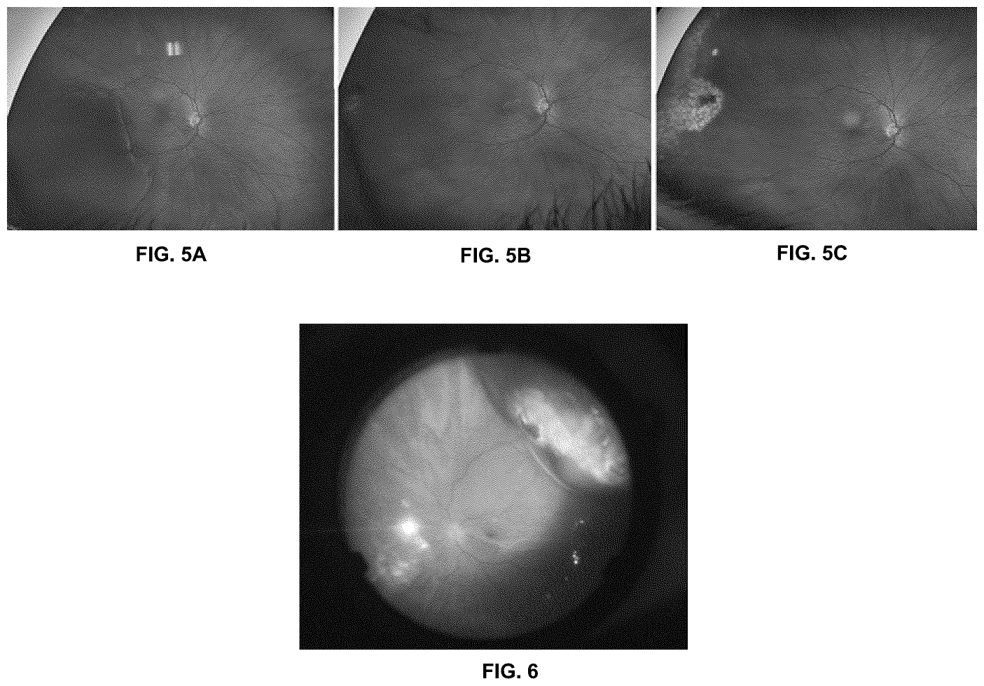

A man in his 50s with pseudophakia presented to St Michael's Hospital, Unity Health Toronto, with a reduced Snellen best-corrected visual acuity of 20/50 OD. FIGS. 5A-5C show longitudinal ultrawide-field photographs of this man with pseudophakia presenting with a RRD in the right eye. FIG. 5A shows a baseline image demonstrating a fovea-involving inferotemporal RRD from 6 to 10 o'clock, with no definitive causative retinal break. Based on the rules by Lincoff and Gieser¹², the break was assumed to be temporal or superior temporal. After discussing treatment options and obtaining written informed consent, sodium hyaluronate 1% (Provisc, Alcon) was injected in the superior temporal quadrant with the patient under subconjunctival anesthesia, using an early prototype that has some similar uses as some of the device embodiments described herein where the prototype included a 30-gauge needle with a custom-made guard that exposed 1 mm of the needle. The custom guard was made using intravenous tubing (Med-RX, Canadian Hospital Specialties Ltd). A syringe loaded with the viscoelastic was coupled to the needle. The injection site in the location of the suspected tear was verified internally with indirect ophthalmoscopy (confirming that the needle was not too deep), and 0.4 mL of viscoelastic was slowly injected transconjunctivally under direct visualization while a dome-shaped choroidal elevation formed. During the injection, the patient had an initial pressure feeling (as did the injecting assistant) as the choroidal bleb was initiated, which lessened and was tolerable during the rest of the procedure as the bleb propagated. Anterior chamber paracentesis was not required because central retinal artery perfusion was confirmed. Although a video of the choroidal convexity forming inside the eye was not available for the in-office procedure, a similar technique was performed intraoperatively in another 2 patients as a combined PPV and ST method, which was used for additional support of inferior and temporal retinal breaks. In particular, FIG. 6 shows a final appearance of the choroidal convexity formed after performing the ST method. The reporting guideline for case series by Kempen was followed.

### Results

The patient was able to resume normal activity with no restrictions the day after the procedure. On the first day after performing the ST method, the macula was completely attached. For example, FIG. 5B shows a first-day post-ST method demonstrating substantial resolution of the retinal detachment with some initial spots of laser retinopexy that were applied to the temporal periphery. A small localized temporal hemorrhage was noted near the injection site.

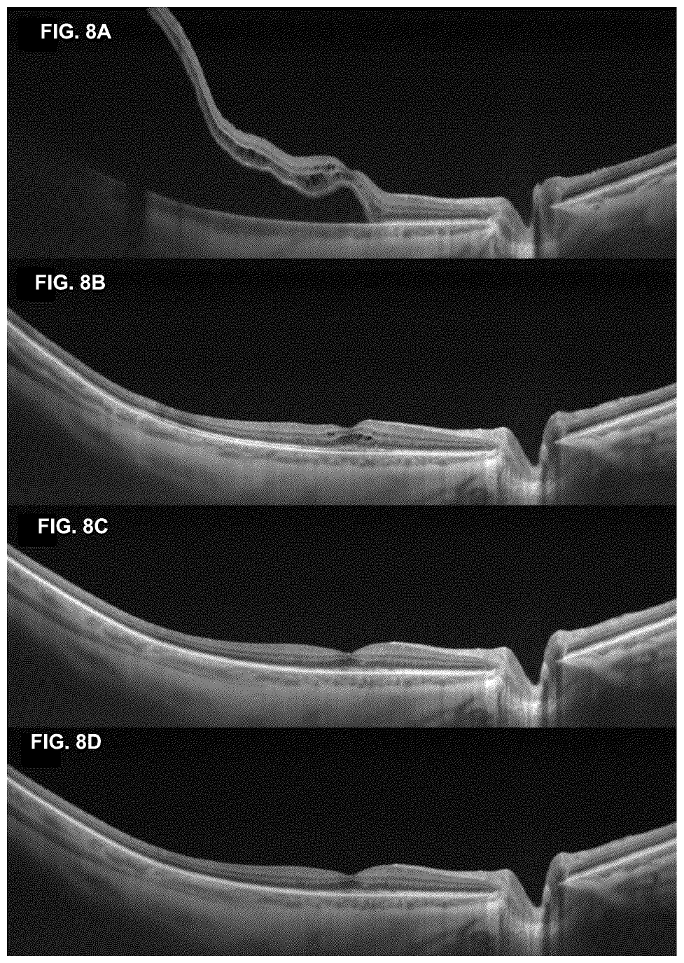

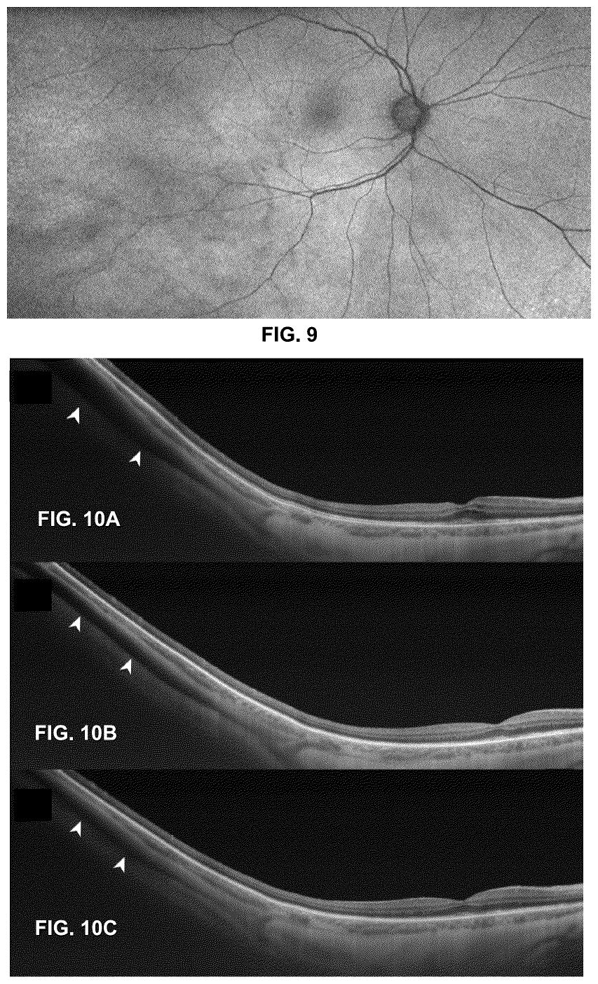

Longitudinal swept-source optical coherence tomography (SS-OCT) demonstrated reattachment with rapid recovery of foveal external limiting membrane and ellipsoid zone integrity (see FIGS. 8A-8D). The patient progressed through the stages of reattachment with no anatomic abnormalities: FIG. 8A shows a baseline scan. FIG. 8B shows a postoperative day 1 scan with significant improvement in the outer retinal corrugations and cystoid macular edema (Stage 2). FIG. 8C shows a postoperative day 2 scan demonstrating contact of the retina with the retinal pigment epithelium (Stage 3). FIG. 8D shows a postoperative day 3 scan demonstrating a deturgescence of the bacillary layer (Stage 4). On postoperative day 5 (no figure shown), the patient demonstrated improved integrity of the outer retinal bands (Stage 5). The patient achieved complete retinal reattachment with rapid recovery of the external limiting membrane and ellipsoid zone integrity. As shown in FIG. 9, fundus autofluorescence imaging was performed on postoperative day 5 with no signs of retinal displacement on the posterior pole, indicating that the patient achieved a high-integrity retinal reattachment (HIRA).

The SS-OCT scans in the location of the choroidal convexity demonstrated a hyporeflective gap between the sclera and choroid, indicating the location of the viscoelastic in the suprachoroidal space (SCS). For example, FIGS. 10A-10C show longitudinal swept-source optical coherence tomography scans at the temporal macula and temporal mid-periphery demonstrating the location of the ST method on the left side of the image. The arrowheads are pointing to the viscoelastic that is in a hyporeflective space between the sclera and the choroid can be observed, indicating the location of the viscoelastic material in the suprachoroidal space over the first postoperative week. FIG. 10A shows a postoperative day 1 scan, FIG. 10B shows a postoperative day 3 scan and FIG. 10C shows a postoperative day 5 scan. A progressive reduction in the height of this hyporeflective gap is observed throughout the post-ST method period (arrowheads).

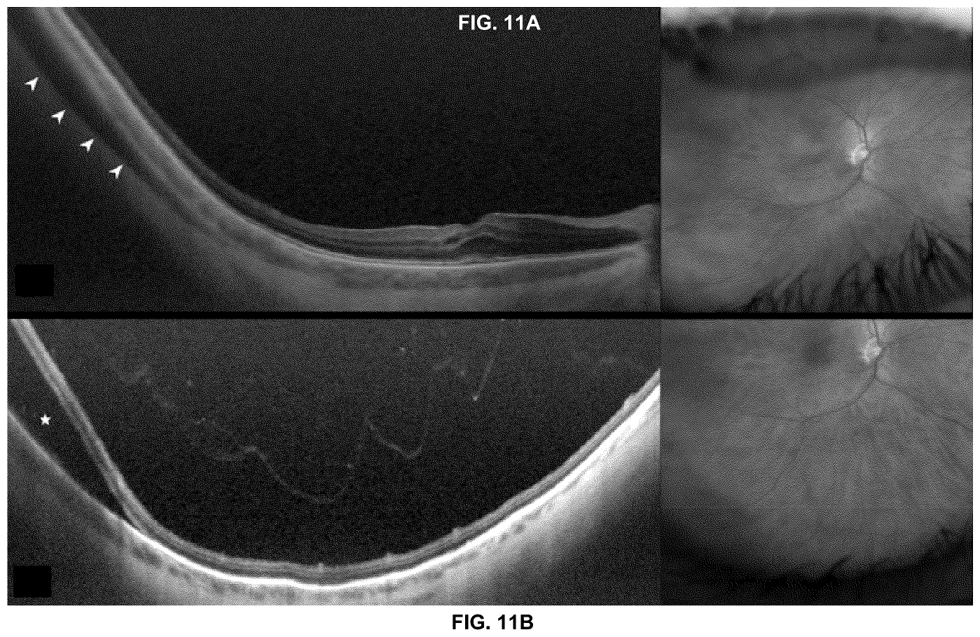

Laser retinopexy was applied in the suspected region of the tear on the first postoperative day (see FIGS. 5B and 5C). For example, FIG. 5C shows third-day post-ST method demonstrating complete laser retinopexy barricade in the suspected region of the causative retinal break. Mild residual subretinal fluid was observed in the inferior periphery and slowly improved with no open breaks. For example, ultra-wide-field fundus swept-source OCT scans demonstrating complete reattachment of the macular region are shown in FIGS. 11A-11B. In particular, FIG. 11A shows a hyporeflective space between the choroid and sclera (shown by the arrowheads) demonstrating the suprachoroidal viscoelastic material. FIG. 11B shows mild residual inferior subretinal fluid with no outer retinal folds in the extreme inferior periphery (star), representing residual subretinal fluid that slowly resolved over time.

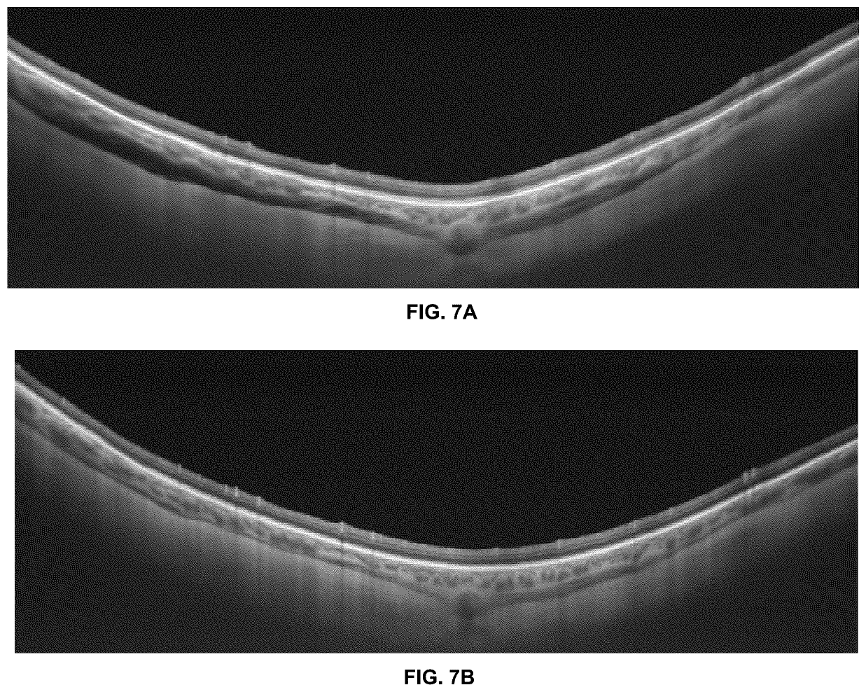

The choroidal convexity reduced in size during the first week (see FIGS. 7A and 7B) and had completely resolved by 2 weeks. FIGS. 7A-7B were obtained by performing longitudinal vertical swept-source optical coherence tomography at the viscopexy injection site. FIGS. 7A-7B demonstrate the progressive reabsorption of the suprachoroidal viscoelastic (hyporeflective space between the choroid and sclera indicated by the arrowheads) from postoperative day 1 (FIG. 7A) to postoperative day 5 (FIG. 7B). The extent of the suprachoroidal viscoelastic was appreciated with a 12×12-mm volume cube performed in the temporal midperiphery. By postoperative day 5, best-corrected visual acuity was 20/25, which remained stable during the first month of follow-up.

### DISCUSSION

The ST method can be performed in the office and seems relatively less invasive compared with prior approaches of SCS viscoelastic injection, although there are risks of choroidal hemorrhage, infection, and inadvertent intraocular injection or retinal perforation. The procedure in the study was performed using an early prototype with a custom 30-gauge needle guard using intravenous tubing that exposed 1 mm of the needle and allowed injection of viscoelastic into the SCS from a syringe that was loaded with the viscoelastic and fluidically coupled to the guarded needle. During the injection, if the needle is intrascleral, there is significant resistance that is overcome as the needle is slightly advanced into the SCS with additional pressure.

From the study, the inventor believes that performing the ST method in-office may be a reasonable approach in select patients. This procedure may be well suited for acute RRDs without proliferative vitreoretinopathy and may be preferable in cooperative patients with breaks within 1 clock hour. It may be particularly beneficial for inferior breaks where pneumatic retinopexy maybe less likely to succeed; however, the procedure may also be performed according to the teachings herein for superior breaks because the lack of tamponade and positioning requirements are significant advantages of the ST method. In addition, the ST method may be performed in combination with PPV or pneumatic retinopexy for additional support of retinal breaks. Sodium hyaluronate 2.3% (Healon 5, Abbott Medical Optics) may be preferable in some cases because it remains in the SCS for up to 3 weeks and retains a useful effect for at least 7 to 10 days.

The benefits of performing this technique in accordance with the teachings herein include the complete natural reabsorption of the viscoelastic agent, the reduced invasiveness with the injection through a small-gauge needle, and the fact that these substances are immunologically inert. Longer-acting viscoelastic agents may be superior. In addition, some of the complications associated with traditional SB surgery may be avoided.

In one aspect, in accordance with the teachings herein, there is provided at least one embodiment of a device for use in a patient with an eye having a rhegmatogenous retinal detachment (RRD) or a retinal tear, wherein the device comprises: a probe including: a main body having a longitudinal axis and a distal end that is angled/curved or straight with respect to the longitudinal axis; an injection conduit for receiving a treatment fluid for injection into the suprachoroidal space (SCS) of the eye for treating the RRD or the retinal tear; a needle disposed at the distal end of the probe, the needle having a needle conduit that is fluidically coupled to the injection conduit for injecting the treatment fluid into the SCS of the eye; and an actuator that is controllable by a user for causing the treatment fluid to move from the injection conduit through the needle conduit into the SCS of the eye.

In another aspect, in accordance with the teachings herein there is provided at least one embodiment of a device for injecting or draining fluid into/from an eye of a patient, wherein the device comprises: a probe including: a main body having a longitudinal axis and a distal end that is angled or straight with respect to the longitudinal axis of the main body; one or more probe conduits for moving the fluid through the probe; a distal curved end portion having a lower surface and a portion of the lower surface is placed adjacent to a surface of the eye during use; and an extendable needle disposed at the distal end of the probe, the needle having a needle conduit that is fluidically coupled to the one or more probe conduits for injecting or draining the fluid into/from the eye and the needle being extended approximately perpendicularly away from an axis of the distal end portion of the probe for penetrating the sclera.

In at least one embodiment, the device comprises a fluid actuator that is controllable by a user for causing the fluid to move between the one or more probe conduits and the eye through the needle conduit.

In at least one embodiment, the distal end portion of the probe has a predetermined radius of curvature, arc length and arc angle for accessing a desired location on the eye surface during use where the radius of curvature approximately matches a radius of curvature of the eye or sclera where the needle is inserted.

In at least one embodiment, the distal end portion has a boss on the lower surface so that the user is aware of where the needle will extend from the probe.

In at least one embodiment, the needle is extended out of the boss during use.

In at least one embodiment, the distal end portion of the probe is rigid.

In at least one embodiment, the distal end portion is flexible to adjust a radius of curvature of the lower surface of the distal end portion.

In at least one embodiment, the arc angle of the distal end portion is between about 15 degrees to about 60 degrees.

In at least one embodiment, the eye has a rhegmatogenous retinal detachment (RRD) or a retinal tear, the device is adapted for injecting the fluid through the injection conduit and the needle conduit into a suprachoroidal space (SCS) of the eye for treating the RRD or the retinal tear.

In at least one embodiment, the fluid is injected to create a choroidal buckle to treat the RRD or the retinal tear.

In at least one embodiment, the one or more probe conduits comprise an injection conduit for injecting the fluid into the eye and a drainage conduit for draining the fluid from the eye, and the needle has a needle position that is adjustable between a drainage position for fluidically coupling the drainage conduit to the needle conduit for draining fluid from the subretinal space or other location in the eye and an injection position for fluidically coupling the injection conduit to the needle conduit for injecting the treatment fluid into the SCS or other locations of the eye including the subretinal space, the choroid or intravitreal spaces.

In at least one embodiment, the device comprises a fluid actuator that is controllable by a user for causing the fluid to move through the needle conduit from the injection conduit into the eye and/or from the eye into the drainage conduit.

In at least one embodiment, the probe comprises a needle actuator that is user adjustable for adjusting the needle position between the draining position and the injection position.

In at least one embodiment, the probe comprises a needle position indicator to indicate to the user the needle position.

In at least one embodiment, during drainage a tip of the needle is adapted to extend from about 1.5 mm to about 3 mm into the eye to reach a subretinal location of the eye.

In at least one embodiment, at the injection position the needle is adapted to extend about 0.3 mm to about 1.5 mm into the eye for a first injection into the SCS or the needle is adapted to extend from about 1 mm to about 2 mm into the eye for a second injection into the SCS.

In at least one embodiment, the fluid actuator and/or needle actuator are each coupled to a pedal and/or a switch that are configured to be controlled by the user.

In at least one embodiment, the needle comprises a circumferential aperture that is fluidically coupled to the drainage conduit when the needle position is the drainage position and the circumferential aperture is fluidically coupled to the injection conduit when the needle position is the injection position.

In at least one embodiment, the drainage conduit comprises a drainage cylinder having a distal end that is fluidically coupled with a circumferential pore of the needle conduit when the needle position is the draining position and the injection conduit comprises an injection cylinder that is located in the draining cylinder and has a distal end that is fluidically coupled with the circumferential pore of the needle conduit when the needle position is the injection position.

In at least one embodiment, the probe has a form factor that allows the probe to be handheld, and the distal end of the probe is shaped to enable the user to indent the sclera of the eye during use, the distal end of the probe being shaped as a scleral depressor.

In at least one embodiment, the device has at least one sensor to allow the user to determine a location of the retinal tear or retinal detachment or localize the site of drug delivery in the eye during use.

In at least one embodiment, the probe comprises at least one sensor that is any combination of an electrical impedance sensor, a mechanical resistance sensor, a pressure sensor, and a flow sensor to measure an electrical impedance, insertion resistance and/or injection resistance where the electrical impedance, insertion resistance or injection resistance at approximately a position of a tip of the needle is used to determine when the tip of the needle is in the sclera or SCS of the eye.

In at least one embodiment, the device further comprises a guidance light source that is adapted to generate a guidance light beam having one or more predetermined wavelengths to indicate when a tip of the needle penetrates into different layers of the eye by changes in light intensity.

In at least one embodiment, the device further comprises a light guidance sensor to sense a reflection of the guidance light beam to generate location data based on where the sensed reflected light beam had a different light intensity when a location of the needle tip is in the sclera or SCS of the eye.

In at least one embodiment, the device further comprises a guidance tool that is optically coupled to the distal end of the probe to aid the user in positioning a distal tip of the needle at the break location, wherein the guidance tool includes a light source for illuminating the distal tip of the needle.

In at least one embodiment, the device further comprises a guidance tool that is optically coupled to the distal end of the probe to aid the user in positioning a distal tip of the needle at the break location, wherein the guidance tool is an Optical Coherence Tomography (OCT) device an Optical Coherence Elastography (OCE) device, an endoscope imaging device, a light intensity sensing device, a light scattering sensing device, a light wavelength sensing device, a laser sensing device, or a light polarization sensing device.

In at least one embodiment, the device further comprises a guidance tool that includes generating acoustic waves that are emitted from the distal end of the probe to aid the user in positioning a distal tip of the needle at the break location, wherein the guidance tool is an ultrasound imaging device or an acoustic reflection measuring device.

In at least one embodiment, the device further comprises a control unit that includes: a display; a memory unit for storing software instructions for performing one or more functions; a device interface for receiving measurement data from the probe; a processor that is communicatively couped to the memory unit, the interface and the display, the processor being configured to perform the one or more functions when executing the software instructions, the one or more functions including: receiving the measurement data; and displaying at least a portion of the measurement data on the display.

In at least one embodiment, the measurement data includes any combination of insertion resistance, injection resistance, and/or electrical impedance to determine needle depth penetration for indicating the location of the needle tip in the eye.

In at least one embodiment, the device includes a pump that is controllable by the processor, an injection port to receive an injection tube that is fluidically coupled to the injection conduit of the probe, and an internal injection conduit that is fluidically coupled to the injection port and the pump and the pump is connected to a fluid source, wherein during injection the processor is configured to send a pump control signal to the pump to create an injection pressure to move the fluid from the fluid source to the eye.

In at least one embodiment, the injection fluid may also be pre-loaded into the device or the device may be filled with the injection fluid immediately before use.

In at least one embodiment, an amount of the injection pressure is set to a predetermined injection pressure level which is adjustable between about 20 mmHg to about 70 mmHg.

In at least one embodiment, the device includes a pump that is controllable by the processor, at least one aspiration port to receive at least one aspiration tube that is fluidically coupled to the drainage conduit of the probe, and an internal drainage conduit that is fluidically coupled to the at least one aspiration port and the pump, wherein the pump is connected to a drainage container, and wherein during draining the processor is configured to send a pump control signal to the pump to create an aspiration pressure to move the drainage fluid from the eye to the drainage container.

In at least one embodiment, the drainage fluid may be collected in a drainage tube within the device.

In at least one embodiment, the amount of aspiration pressure is set to a predetermined aspiration pressure level, which is adjustable from 0 mmHg to about 700 mmHg.

In at least one embodiment, the actuator is coupled to the control unit so that during operation the fluid actuator sends an actuator control signal to the processor when actuated by the user to control injection or drainage at the probe.

In at least one embodiment, the probe includes an optical conduit that is disposed along the injection conduit and the control unit comprises a light port for receiving an optical fiber that is optically coupled to the optical conduit, and the control unit comprises an internal light conduit that is optically coupled to the light port and the light source.

In at least one embodiment, the device may also contain its own internal light source, eliminating the need for an external light source.

In at least one embodiment, the probe includes an optical conduit that is disposed along the injection conduit and the control unit includes the OCT device, the Optical Coherence Elastography (OCE) device, the endoscope imaging device, the light intensity sensing device, the light scattering sensing device, the light wavelength sensing device, or the light polarization sensing device, a light port for receiving an optical fiber that is optically coupled to the optical conduit, an internal light conduit that is optically coupled to the light port, and a display for displaying one or more images provided by the OCT device, the Optical Coherence Elastography (OCE) device, the endoscope imaging device, the light intensity sensing device, the light scattering sensing device, the light wavelength sensing device, or the light polarization sensing device.

In at least one embodiment, the probe includes an ultrasound transducer that is disposed at a distal end of the probe and the control unit comprises the ultrasound imaging or acoustic reflection measuring device that is coupled to the ultrasound transducer for receiving ultrasound signals or acoustic reflection measurements, processing the received ultrasound signals to generate ultrasound images or processing the received acoustic reflection measurements, and displaying the ultrasound images or the processed acoustic reflection measurements on the display.

In at least one embodiment, the processor is configured to display operating parameters including any combination of the measured resistance, the injection pressure, the aspiration pressure and location data on the display.

In at least one embodiment, the control unit comprises a speaker or a vibrator that is communicatively coupled to the processor and the processor is configured to generate audio signals or vibrations and output the audio signals via the speaker or vibrations via the vibrator, where the audio signals include speech, tones or beeps and the audio signals or the vibrations correspond to the device operating parameters and/or measurement data including any combination of the injection pressure, and the aspiration pressure or other data.

In at least one embodiment, a vibrator may be included to provide vibration feedback in the probe to provide tactile feedback, that is preferably gentle (e.g., small in amplitude).

In at least one embodiment, the probe further comprises a flange at the distal end of the probe to maintain a position of the distal end of the probe and the tip of the needle relative to a surface of the eye.

In at least one embodiment, the flange further comprises one or more pressure sensors in communication with the control unit to measure the pressure at one or more points on the flange and the control unit is configured to provide visual or auditory output indicative of the measured pressure.

In at least one embodiment, the device further comprises a variable coupler at the distal end portion of the probe that is adapted to dampen pressure applied by the distal end portion of the probe to the eye when a pressure or force applied to the eye during use is higher than a threshold.

In at least one embodiment, the variable coupler comprises a pressure release valve which releases pressure when the applied pressure to the eye during use is higher than the threshold or the variable coupler comprises a force damper when force applied to the eye during use is higher than the threshold.

In at least one embodiment, the probe is telescopic to extend the distal end portion.

In another aspect, in accordance with the teachings herein, there is provided at least one embodiment of a method for treating a patient with an eye having a rhegmatogenous retinal detachment (RRD) or a retinal tear, wherein the method comprises: setting up an ocular treatment device having a probe and a needle, the needle having an adjustable needle position; examining the patient's eye; using the device with the needle retracted to depress and find one or more retinal breaks; advancing a needle position of the needle to an injection position to move the needle towards a suprachoroidal space (SCS) of the patient's eye; confirming a position of a tip of the needle using a guidance tool; and injecting a treatment fluid so that the treatment fluid enters the SCS of the patient's eye.

In at least one embodiment, the injection is started just prior to the tip of the needle entering the SCS.

In at least one embodiment, the injection is started while the needle is advancing through the sclera and a decrease in injection resistance is used to indicate when the needle has passed through the sclera and entered the SCS.

In at least one embodiment, the needle is in the injection position the tip of the needle extends about 0.3 to about 1.5 mm.

In at least one embodiment, the method further comprises performing the method again to provide additional injections of the treatment fluid to the patient on the same day or a different day in the future.

In at least one embodiment, for the second injection the tip of the needle extends about 1 mm to about 2 mm.

In at least one embodiment, the treatment fluid is a viscoelastic fluid, an inert gas or air, a hydrogel, an extended release implant, or a drug solution.

In at least one embodiment, the drug solution includes preservatives, or is preservative free, and/or the drug solution is used for antibody treatments, gene therapy, steroid treatment, or other pharmacological therapy.

In at least one embodiment, the viscoelastic agent comprises a hyaluronic acid, a cross-linked hyaluronic acid, sodium hyaluronate 1%-2.3%, and dissolvable or non-dissolvable hydrogel spacers.

In at least one embodiment, when it is determined to drain fluid from the subretinal space of the patient's eye, the method further comprises: advancing the needle position to a drainage position; and performing active or passive fluid drainage from the subretinal space before or after injection into the SCS.

In at least one embodiment, in the drainage position the tip of the needle extends about 1.5 mm to about 3 mm into the eye and into the subretinal space without touching the retina.

In at least one embodiment, a controllable needle actuator is used for advancement and retraction of the needle and the needle actuator is user-controlled.

In at least one embodiment, a fluid actuator is used for the injection of the treatment fluid and the fluid actuator is user-controlled.

In at least one embodiment, the guidance tool comprises any combination of a light source, Optical Coherence Tomography (OCT) device, an Optical Coherence Elastography (OCE) device, an endoscope imaging device, a light intensity sensing device, a light scattering sensing device, a light wavelength sensing device, or a light polarization sensing device, an ultrasound imaging device, an acoustic reflection measurement device and a handheld lens.

In at least one embodiment of the method, the device is defined according to any of the embodiments described herein.

In another aspect, in accordance with the teachings herein, there is provided at least one embodiment of a use of a device having a needle for treating an eye of a patient, wherein the device is defined according to any of the embodiments described herein.

In another aspect, in accordance with the teachings herein, there is provided a method of injecting or draining fluid into/from an eye of a patient, wherein the method comprises: positioning a probe that is defined according to appropriate embodiments described herein, such that a lower surface of a distal end portion of the probe indents a surface of the eye to be treated; extending a tip of a needle from the lower surface of the distal end of the probe into the eye at a desired depth, the needle being extended approximately perpendicularly from an axis of the distal end portion of the probe; determining a depth of the needle tip using at least one sensor and/or a visual guidance tool; injecting or draining fluid into/from the eye when the needle tip is at a desired depth; and removing the needle from the eye.

In at least one embodiment, in accordance with the teachings herein, there is provided a self-contained handheld device for injecting or draining fluid into/from an eye of a patient, wherein the device is shaped as a probe and comprises: a main body having a longitudinal axis and a distal end having a longitudinal axis that is angled or straight with respect to the longitudinal axis of the main body; one or more probe conduits for moving the fluid through the probe; a distal end portion having a lower surface and a portion of the lower surface is placed adjacent to a surface of the eye during use; an extendable needle disposed at the distal end of the probe, the needle having a needle conduit that is fluidically coupled to the one or more probe conduits for injecting or draining the fluid into/from the eye and the needle being extended approximately perpendicularly away from a longitudinal axis of the distal end portion of the probe for penetrating the sclera; a needle actuator and needle actuator control for extending or retracting the needle; and a fluid actuator and a fluid actuator control for controlling injection or drainage of the fluid.

In at least one embodiment, the device includes an input button that is configured to allow a user to configure the device to operate in injection mode or drainage mode.

In at least one embodiment, the device includes at least one sensor for measuring data related to a penetration depth of a tip of the needle during use.

In at least one embodiment, the device comprises a light source for generating a guidance light beam that is transmitted from the distal end portion of the probe towards the eye during use.

In at least one embodiment, the device comprises a microcontroller for controlling operation the device, the microcontroller is located in the probe.

In at least one embodiment, the device comprises a power source.

In at least one embodiment, the device includes a treatment fluid container for providing treatment fluid during injection.

In at least one embodiment, the device includes a drainage tube for receiving drained fluid during use and a drain port that is coupled to the drainage tube for removing the drained fluid.

In at least one embodiment, the lower surface of the distal curved end portion has a radius of curvature that approximates a radius of curvature of the eye or sclera at needle penetration and an arc length that is predetermined based on the location on the eye where needle penetration is to be performed.

While the applicant's teachings described herein are in conjunction with various embodiments for illustrative purposes, it is not intended that the applicant's teachings be limited to such embodiments. On the contrary, the applicant's teachings described and illustrated herein encompass various alternatives, modifications, and equivalents, without generally departing from the embodiments described herein. For example, while the teachings described and shown herein may comprise certain elements/components and steps, modifications may be made as is known to those skilled in the art. For example, selected features from one or more of the example embodiments described herein in accordance with the teachings herein may be combined to create alternative embodiments that are not explicitly described. For example, as will be clear to a person having ordinary skill in the art, the devices and methods described herein may be implemented using other additional combinations and permutations of the various features and functions presented including shape, form factor, needle position or orientation, conduits, adjustments, controls, actuators, indicators, guidance elements, fluids and other materials, procedure steps, and applications. All values and sub-ranges within disclosed ranges are also disclosed. The subject matter described herein intends to cover and embrace all suitable changes in technology.

### REFERENCES

1. Williams G A, Aaberg T A Jr. Techniques of scleral buckling. In: Ryan S J, Wilkinson C P, eds. Retina. Vol. 3. 4th ed. Elsevier Mosby; 2006:2035-2207.
2. Moinuddin O, Abuzaitoun R O, Hwang M W, et al. Surgical repair of primary non-complex rhegmatogenous retinal detachment in the modern era of small-gauge vitrectomy. BMJ Open Ophthalmol 2021; 6 (1): e000651. doi: 10.1136/bmjophth-2020-000651.
3. Heimann H, Hellmich M, Bornfeld N, Bartz-Schmidt K U, Hilgers R D, Foerster M H. Scleral buckling versus primary vitrectomy in rhegmatogenous retinal detachment (SPR Study): design issues and implications. SPR Study report no. 1. Graefes Arch Clin Exp Ophthalmol 2001; 239 (8): 567-574. doi: 0.1007/s004170100319.
4. Hillier R J, Felfeli T, Berger A R, et al. The Pneumatic Retinopexy versus Vitrectomy for the Management of Primary Rhegmatogenous Retinal Detachment Outcomes Randomized Trial (PIVOT). Ophthalmology 2019; 126 (4): 531-539. doi: 10.1016/j. ophtha.2018.11.014.
5. Brosh K, Francisconi C L M, Qian J, et al. Retinal displacement following pneumatic retinopexy vs pars plana vitrectomy for rhegmatogenous retinal detachment. JAMA Ophthalmol 2020; 138 (6): 652659. doi: 10.1001/jamaophthalmol.2020.1046.
6. Francisconi C L M, Marafon S B, Figueiredo N A, et al. Retinal displacement after pneumatic retinopexy versus vitrectomy for rhegmatogenous retinal detachment (ALIGN). Ophthalmology 2022; 129 (4): 458-461. doi: 10.1016/j.ophtha.2021.12.007.
7. Lee W W, Bansal A, Sadda S R, et al. Outer retinal folds after pars plana vitrectomy vs. pneumatic retinopexy for retinal detachment repair: post hoc analysis from PIVOT. Ophthalmol Retina 2022; 6 (3): 234-242. doi: 10.1016/j.oret.2021.09.001.
8. Muni R H, Felfeli T, Sadda S R, et al. Postoperative photoreceptor integrity following pneumatic retinopexy vs pars plana vitrectomy for retinal detachment repair: a post hoc optical coherence tomography analysis from the pneumatic retinopexy versus vitrectomy for the management of primary rhegmatogenous retinal detachment outcomes randomized trial. AMA Ophthalmol 2021; 139 (6): 620-627. doi: 10.1001/jamaophthalmol. 2021.0803.
9. Bansal A, Naidu S C, Marafon S B, et al. Retinal displacement after scleral buckle versus combined buckle and vitrectomy for rhegmatogenous retinal detachment: ALIGN scleral buckle versus pars plana vitrectomy with scleral buckle. Ophthalmol Retina Published online May 20, 2023. doi: 0.1016/j.oret. 2023.05.012.
10. Mckay B R, Bansal A, Kryshtalskyj M, Wong D T, Berger A, Muni R H. Evaluation of Subretinal fluid Drainage Techniques During Pars Plana Vitrectomy for Primary Rhegmatogenous Retinal Detachment-ELLIPSOID Study. Am J Ophthalmol 2022; 241:227-237. doi: 0.1016/j.ajo.2022.05.008.
11. Farahvash A, Marafon S B, Juncal V R, Figueiredo N, Ramachandran A, Muni R H. Impact of tamponade agent on retinal displacement following pars plana vitrectomy for rhegmatogenous retinal detachment repair: a computer simulation model. Acta Ophthalmol 10.1111/aos.15118.
12. Lincoff H, Gieser R. Finding the retinal hole. Arch Ophthalmol 971; 85 (5): 565-569. doi: 0.1001/archopht. 1971.00990050567007.
13. Kempen J H. Appropriate use and reporting of uncontrolled case series in the medical literature. Am J Ophthalmol 2011; 151 (1): 7-10.e1. doi: 0.1016/j. ajo.2010.08.047.

1. A device for injecting or draining a fluid into or from an eye, wherein the device comprises:
a probe comprising:
a main body having a distal end portion;
a needle that extends and retracts from an exit location on a side surface of the distal end portion, the needle having a needle conduit; and
one or more probe conduits for moving the fluid through the probe, the one or more probe conduits being fluidically coupled to the needle conduit;
wherein during use, a portion of the side surface having the exit location is placed adjacent to a surface of the eye and the needle is extended to penetrate into the eye, and the fluid is injected or drained through the needle conduit.

2. The device of claim 1, wherein the needle is configured to exit the probe substantially perpendicular to a tangent of the side surface at the exit location.

3. The device of claim 1, wherein the side surface is concave with a radius of curvature that approximately matches a radius of curvature of the sclera.

4. The device of claim 1, wherein the longitudinal axis of the distal end portion is at an angle to a longitudinal axis of the main body.

5. The device of claim 1, wherein the device comprises a needle actuator that is coupled to the needle and controllable for causing the needle to extend and retract.

6. The device of claim 1, wherein the device comprises a fluid actuator that is coupled to the needle and controllable for causing the fluid to move between the one or more probe conduits and the eye through the needle conduit.

7. The device of claim 1, wherein the side surface has a boss at the exit location and the needle is configured to extend and retract through the boss, or the side surface has a boss adjacent the exit location and the needle is configured to extend and retract adjacent to the boss.

8. The device of claim 1, wherein the one or more probe conduits comprise an injection conduit and a drainage conduit, and the probe has a coupling that is adjustable between fluidically coupling the drainage conduit to the needle conduit and fluidically coupling the injection conduit to the needle conduit.

9. The device of claim 1, wherein the device further comprises a guidance light source that is adapted to generate a guidance light beam for illumination or to indicate when a tip of the needle penetrates into different layers of the eye by changes in transmitted or reflected light.

10. The device of claim 1, wherein the device further comprises at least one guidance tool that is adapted to perform a measurement to determine a position of a tip of the needle and/or a target injection or drainage site in the eye.

11. The device of claim 1, wherein the device further comprises a control unit that is contained in the probe or remote from the probe, the control unity comprising:
a display that is optional;
a memory unit for storing software instructions for performing one or more functions;
a device interface for receiving measurement data and transmitting control signals for operation of the device;
a speaker or vibrator to generate audio signals or vibrations corresponding to device operating parameters and/or the measurement data, where the speaker or vibrator are optional;
a processor that is communicatively coupled to any of the memory unit, the interface, the speaker or vibrator, and the display, the processor being configured to perform the one or more functions when executing the software instructions, the one or more functions including:
receiving the measurement data;
transmitting the control signals;
generating the audio signals or vibrations; and
displaying at least a portion of the measurement data on the display; and
a power source for providing power to components of the device.

12. The device of claim 1, wherein the device includes a pump fluidically coupled to the one or more probe conduits that is controllable to create an injection pressure when the fluid is injected into the eye or a drainage pressure when the fluid is drained from the eye.

13. The device of claim 1, wherein the probe further comprises a flange and/or a variable coupler at the exit location to maintain a position or a pressure between the side surface and the surface of the eye.

14. The device of claim 13, wherein the flange and/or the variable coupler further comprises one or more sensors to measure a position and/or a pressure at one or more points between the side surface of the distal end portion of the probe and the surface of the eye.

15. The device of claim 1, further comprising an injection fluid container and/or a drainage fluid container coupled to the one or more probe conduits.

16. The device of claim 1, wherein the needle is adapted to extend to a depth within a suprachoroidal, subretinal, or intravitreal space of the eye.

17. The device of claim 1, wherein when the eye has a rhegmatogenous retinal detachment (RRD) or a retinal tear and the device is adapted for injecting the fluid into a suprachoroidal space of the eye to create a choroidal buckle for treating the RRD or the retinal tear.

18. The device of claim 1, wherein the fluid comprises a treatment fluid including any combination of a drug, a gene therapy, an extended release implant, a viscoelastic, a hydrogel, and a gas.

19. A method for injecting or draining a fluid into or from an eye, wherein the method comprises:
placing a side surface of a distal end portion of a probe adjacent to a surface of the eye, the probe having a needle with a needle conduit and the needle is retracted;
extending the needle from an exit location on the side surface of the distal end portion of the probe to penetrate the eye; and
injecting or draining fluid between the probe and the eye through the needle conduit.

20. The method of claim 19, wherein the method comprises extending the needle substantially perpendicular to a tangent of the side surface of the probe at the exit location.

21. The method of claim 19, wherein the side surface of the distal end portion of the probe is concave with a radius of curvature that approximately matches a radius of curvature of the sclera.

22. The method of claim 19, wherein a longitudinal axis of the distal end portion is at an angle to a longitudinal axis of the main body.

23. The method of claim 19, wherein the method comprises using a needle actuator for controlling extension and retraction of the needle.

24. The method of claim 19, wherein the method comprises using a fluid actuator for controlling injection and drainage of the fluid.

25. The method of claim 19, wherein the method comprises using a guidance light beam and/or a measurement made by a guidance tool to determine a position of a tip of the needle and/or a target injection or drainage site in the eye.

26. The method of claim 19, wherein a control unit that is integral with or separate from the probe is used to display measurement data from the probe, transmit control signals to the probe, and/or generate audio signals or vibrations corresponding to device operating parameters and/or the measurement data.

27. The method of claim 19, wherein the method comprises extending the needle into a suprachoroidal, a subretinal, or an intravitreal space of the eye.

28. The method of claim 19, wherein the fluid comprises a treatment fluid including any combination of a drug, a gene therapy, an extended release implant, a viscoelastic, a hydrogel, and a gas.

29. The method of claim 19, wherein the eye has a rhegmatogenous retinal detachment (RRD) or a retinal tear and the method comprises injecting the fluid is into a suprachoroidal space (SCS) of the eye to create a choroidal buckle for treating the RRD or the retinal tear.
